(19) United States
(12) Patent Application Publication
(10) Pub. No.: US 2006/0177384 A1
(43) Pub. Date: Aug. 10, 2006

(54) Sialagogue coatings for interproximal devices

(76) Inventor: Dale G. Brown, Wharton, TX (US)

Correspondence Address: BANNER & WITCOFF, LTD., 28 STATE STREET, 28th FLOOR, BOSTON, MA, 02109-9601, US

(21) Appl. No.: 11/349,042

(22) Filed: Feb. 7, 2006

Related U.S. Application Data
(63) Continuation-in-part of application No. 11/118,911, filed on Apr. 29, 2005.
(63) Continuation-in-part of application No. 10/334,089, filed on Dec. 30, 2002, now Pat. No. 7,017,591.
(63) Continuation-in-part of application No. 10/073,682, filed on Feb. 11, 2002, now Pat. No. 7,025,986.
(63) Continuation-in-part of application No. 10/331,800, filed on Dec. 30, 2002.
(63) Continuation-in-part of application No. 10/005,902, filed on Dec. 4, 2001.

(51) Int. Cl. A61K 8/73; A61K 8/89

(52) U.S. Cl. 424/49

(57) ABSTRACT

Biofilm therapy interproximal devices coated with a sialagogue, a wax/sialagogue, a surfactant/sialagogue, or an emulsion/sialagogue. wherein said devices are suitable for: (c) controlling, disrupting and physically removing biofilms, while (d) increasing and prolonging saliva flow.

### CROSS-REFERENCE TO RELATED APPLICATIONS

This application is a continuation-in-part of the following copending applications: U.S. patent application Ser. No. 10/005,902, filed Dec. 4, 2001 entitled “Biofilm Therapy Process and Elements”; U.S. patent application Ser. No. 10/331,800, filed Dec. 30, 2002, entitled, “Coated Micromesh Dental Devices Overcoated with Imbedded Particulate”; U.S. patent application Ser. No. 10/073,682, filed 11 Feb. 2002, entitled, “Micromesh Interproximal Devices”; U.S. patent application, Ser. No. 10/334,089, filed Dec. 30, 2002, entitled, “Particulate Coated Monofilament Devices; and U.S. patent application, Ser. No. 11/118,911, filed Apr. 29, 2005, entitled, “Coated Monofilament Oriented HDPE Dental Tapes”. The disclosures of these applications are hereby incorporated herein by reference.

### FIELD OF INVENTION

The field of the present invention relates to specially treated interproximal dental devices. These interproximal dental devices include a variety of medical devices suitable for cleaning spaces between teeth and below the gumline. In addition to dental flosses and dental tapes, other suitable interproximal dental devices include flossers, dental picks, dental massagers, proxy brushes, interdental irrigators and the like. All of these biofilm therapy, interproximal dental devices can be treated with saliva soluble coatings or, in the case of interproximal irrigators the saliva soluble ingredients can be added to the liquid irrigant. These treatments contain: (a) sialagogues, (b) wax/sialagogue mixtures, or (c) various biofilm-responsive substances containing sialagogues. These devices are suitable for physically controlling, disrupting and removing biofilms, while delivering: sialagogues or biofilm-responsive coatings containing sialagogues to interproximal surfaces to disrupt and control biofilms, to simultaneously increase and prolong saliva flow and to simultaneously deliver anti-inflammatory properties subgingivally and interproximally.

### BACKGROUND OF THE INVENTION

Dental floss is defined in Webster's New World Dictionary, 1983, as “ . . . thread for removing food particles between the teeth.”

The concept of using dental floss for cleansing interproximal spaces appears to have been introduced by Parmly in 1819, Practical Guide to the Management of Teeth, Cullins & Croft Philadelphia, Pa. Numerous types of floss were developed and used for cleaning interproximal and subgingival surfaces, until finally in 1948 Bass established the optimum characteristics of dental floss, Dental Items of Interest, 70, 921-34 (1948).

Bass cautioned that dental floss treated with sizing, binders and/or wax produces a “cord” effect as distinguished from the desired “spread filament effect”. This cord effect reduces flossing efficiency dramatically and visually eliminates splaying (i.e., the flattening and spreading out of filaments) necessary to achieve the required interproximal and subgingival mechanical cleaning. This cleaning is then required to be followed by the entrapment and removal of loosened: debris, plaque and microscopic materials from interproximal spaces by the “spread” floss as it is removed from between teeth.

Dental flosses, dental tapes, dental flossers, dental picks, dental massagers, proxy brushes and interdental irrigators are described below.

All of the foregoing devices can be treated with saliva soluble substances containing sialagogues and, once so treated, are included in the biofilm-responsive interproximal devices of the present invention that increase and prolong saliva flow while delivering anti-inflammatory properties interproximally and subgingivally.

Proper use of the various interproximal dental devices such as referenced above is necessary to clean the considerable surface area on the interproximal surfaces of teeth (approximately 40% of total tooth surfaces), as well as subgingival areas, both of which cannot usually be reached by other cleaning methods or agents, e.g., the bristles of a toothbrush, the swishing action of a rinse, or by the pulsating stream from an oral irrigator.

Historically, the purpose of interproximal dental devices was to:
(1) dislodge and remove any decomposing food material, debris, etc., that has accumulated at the interproximal surfaces, which could not be removed by other oral hygiene means, and
(2) dislodge and remove as much as possible the growth of bacterial material (plaque, tartar, calculus . . . eventually to be classified as biofilm) that had accumulated there since the previous cleaning.

Effective interproximal oral hygiene requires that three control elements be maintained by the individual:
(1) Physical removal of stains, plaque and tartar interproximally. This is accomplished in the strongest sense by scraping and abrasion in the dentist's office during prophylaxis, scaling or root planing. Self administered physical procedures are required frequently between visits to the oral care professional and range from tooth brushing with an appropriate abrasive toothpaste through use of the various interproximal dental devices described above, as well as water jet action.
(2) Surfactant Cleaning. This is required to remove: food debris and staining substances before they adhere to the tooth surface; normal dead cellular (epithelial) material which is continually sloughed off from the surfaces of the oral cavity and microbial degradation products derived from all of the above. Research has shown that the primary source of bad breath is the retention and subsequent degradation of dead cellular material sloughed off continuously by the normal, healthy mouth. Besides the obvious hygienic and health benefits related to simple cleanliness provided by surfactants, there is an important cosmetic and sense-of-well-being benefit provided by surfactant cleansing.
(3) Frequency of Cleansing. This is perhaps the most difficult to provide in today's fast-paced work and social environment. Most people recognize that their teeth should be brushed at least 3 times a day and cleaned interproximally at least once a day. The simple fact is that most of the population brush once a day, some brush morning and evening, but precious few carry toothbrush and dentifrice to use the other three or four times a day for optimal oral hygiene. Consumer research suggests that the population brushes an average of about 1.3 times a day. Most surprising, less than 15% of adults use an interdental dental device regularly. Reasons offered for not cleansing interproximally: difficult to do, painful, not effective, doesn't seem to do anything, and leaves a bad taste. There is generally no appreciation by nonflossers for the role plaque (biofilm) buildup plays in exacerbating chronic diseases such as Type II diabetes, heart disease, etc.

Until the introduction of micromesh dental floss as described in copending: U.S. patent application, Ser. No. 10/073,682, entitled, “Micromesh Interproximal Devices”, and U.S. patent application, Ser. No. 11/118,911, entitled, “Coated Monofilament Oriented HDPE Dental Tapes”; there have been only two types of interproximal flossing devices available commercially: multifilament dental flosses and PTFE monofilament dental tapes. Both of the referenced patent applications are incorporated herein by reference.

Examples of multifilament dental flosses are described in the following U.S. Pat. Nos., which are hereby incorporated by reference:
U.S. Pat. Nos. 4,911,927; 4,029,113; 4,610,872; 4,034,771; 5,908,039; 2,667,443; 3,830,246; 1,149,376; 1,069,874; 5,830,495; 2,748,781; 1,138,479; 1,839,486; 1,943,856; 6,080,481; 2,700,636; 3,699,979; 3,744,499; 3,837,351; 4,414,990; 3,330,732; 5,967,155; 5,937,874; 5,505,216; 5,503,842; 5,032,387; 4,950,479; 5,098,711; 1,989,895; 5,033,488; 2,542,518; 2,554,464; 1,285,988; 1,839,483; 4,151,851; 2,224,489; 2,464,755; 2,381,142; 3,800,812; 3,830,246; 3,897,795; 3,897,796; 4,215,478; 4,033,365; 3,771,536; 3,943,949; 6,016,816; 6,026,829; 5,353,820; 5,557,900; 5,226,435; 5,573,850; 5,560,377; 5,526,831; 5,423,337; 5,220,932; 4,548,219; 3,838,702; 5,904,152; 4,911,927; 5,165,913; and 5,098,711.

Examples of texturized multifilament dental flosses are described in U.S. Pat. No. 5,711,935.

Virtually all natural and man-made fibers and specifically filament yarns, twisted continuous filament yarns and the like can be texturized, including: nylon, polyester, polypropylene and various blends of such fibers and/or filaments. Texturizing can be achieved with false twist, core-bulked, textralized, edge crimping, gear crimped, air jet, crinkle, cotexturizing, etc. type processes or modifications thereof as described in E. Stacy, Hosiery Res. Bull. (1): 1-22, 1957 and the references cited at pp. 20-22 of Textured Yarn Technology, Vol. 1. Monsanto, 1967. See also Textured Yarns. Monsanto, 1962 and Stretch Woven Fabrics and Textured Yarns, Monsanto 1963.

These “bulking” processes can be carried out with various types of texturizing equipment as described at pp. 223-233 of Textured Yarn Technology, Vol. 1, Monsanto, 1967, including for instance: Hobnourn No. 2086, ARCT: FT-V, FT-1, FT-F, FT-3, FT-400, FT-U and FT-415; Carolina UNRAV Purdy-Hodges Yarn Crimper, Whitlin RD-5; U.S. Acme Jumbo & Model I Texturizers; Fujiloft F205 Heberlein High Performance Universal Texturizing Machine, Autotwister TZ21, False Twister F231, Ratti Model TRS, Berliner SDD-Set 280, Two-For-One Twister; Turbo-Twist, Pinion & Turbo-Duo Twist.

The equipment and processes used for texturizing are described in numerous U.S. patents and literature as set forth below, the disclosures of which are hereby incorporated by reference into this specification:
(a) U.S. Pat. Nos. 2,463,620; 2,655,781; 2,463,618; 2,904,952; 2,803,105; 23,803,108; and 2,803,109;
(b) “A survey of the literature and patents relating to Bulked Continuous-Filament Yarns”, E. Hutton and W. J. Morris, The Cotton Silk and Man-Made Fibres Research Assoc., Shirley Inst. Didsbury, Manchester, England (1963);
(c) “Bulk, Stretch and Texture”, The Textile Inst., Manchester England 1966 and the various references cited at pp. 97-104 of Textured Yarn Technology, Vol. 1; Monsanto, 1967;
(d) See also: U.S. Pat. Nos. 2,790,611; 2,919,534; 2,921,358; 2,925,641; 2,931,089; 2,977,661; 2,977,746; 2,977,663; 3,017,684; 3,021,588;

3,025,584; 3,028,653; 3,028,654; 3,035,328; and 3,047,932; British Patent Nos. 772,756; 761,084; 801,147; 802,689; and 839,393;
(e) See also yarns and equipment described at pp. 204-234 in Texturized Yarn Technology, Monsanto, Vol. 1 1967; and
(f) See also U.S. Pat. Nos. Re. 23,824; 2,575,837; 2,505,618; 2,514,557; 2,575,781; 2,575,833; 2,575,837; 2,575,838; 2,575,839; 2,647,285; 2,734,228; 2,734,229; 2,734,251; 2,734,252; 2,740,992; 2,758,357; 2,758,358; 2,760,252; 2,854,701; 2,854,728; 2,854,729; 2,960,729; 2,960,730; 2,997,747; 3,000,059; 3,000,060; 3,016,677; 3,027,108; 2,783,609; 2,852,906; 2,868,967; 2,958,112; 2,994,938; 3,013,379; 3,017,737; 3,043,088; and 3,091,908.

Examples of monofilament dental tapes are described in the following U.S. Pat. Nos., which are hereby incorporated by reference:
U.S. Pat. Nos. Re. 35,439; 3,800,812; 4,974,615; 5,760,117; 5,433,226; 5,479,952; 5,503,842; 5,755,243; 5,845,652; 5,884,639; 5,918,609; 5,962,572; 5,998,431; 6,003,525; 6,083,208; 6,198,830; 6,161,555; 6,027,192; 5,209,251; 5,033,488; 5,518,012; 5,911,228; 5,220,932; 4,776,358; 5,718,251; 5,848,600; 5,787,758; 3,664,915; 3,953,566; 3,962,153; 4,096,227; 4,187,390; 4,256,806; 4,385,093; 4,478,665; 4,776,358; 5,518,012 and 5,765,576.

It is generally accepted that both monofilament and multifilament dental flosses are not “user-friendly” interproximal dental devices, i.e., flossing with either is difficult to do. Flossing is generally associated with pain and bleeding and it results in a bad taste in the mouth. Most market researchers agree that anything that can be done to make flossing more positive should be implemented to encourage more frequent flossing and more wide spread floss and/or tape use. The addition to floss and tape of: full spectrum flavor oils, mouth conditioning substances such as silicones along with cleaners and abrasives that are perceived as “working” as taught by the copending patent applications: “Coated Multifilament Dental Devices Overcoated with Imbedded Particulate” and “Coated Monofilament Dental Devices Overcoated with Imbedded Particulate”, both of which are hereby incorporated herein by reference, are all sources of positive feed back to the flosser that would be considered encouraging and supportive, e.g., “it's doing something.”

Most commercial monofilament and multifilament interproximal devices marketed at the present time contain various coatings of wax or wax like substances that function as: (1) binders for the various multifilament flosses to minimize fraying, (2) lubricants, (3) flavor carriers, and/or (4) fluoride carriers for both monofilament and multifilament devices.

An almost universal shortcoming common to most waxed multifilament dental flosses and monofilament dental tapes is the user perception during flossing that the dental floss or dental tape is “not working” and/or “not cleaning”, etc.

Other suitable interproximal devices include dental flossers, dental picks (stimulators), proxy brushes and interdental irrigators, which are discussed in detail below.

Dental Flossers:

For more than one hundred years there have been attempts to improve and modernize the dental flossing technique by development of holders onto which floss is detachably or permanently secured, and a few hundred patents have already been granted on various flossing devices. These go back to the Shurtleft, U.S. Pat. No. 147,987 of 1874, and Wallace, U.S. Pat. No. 175,795 of 1876. In general, the dental flossers of the prior art have fallen into two basic categories, namely the reusable or permanent dental flossers wherein the customer threads the device with floss and then throws away the floss after use, but retains the flosser; and the disposable dental flossers having a structure to which the floss is permanently attached, the entire device being thrown away after one or more uses and replaced by another similar device.

In general, the dental flossers of the permanent type are, as would naturally be expected, of considerably more complex construction and made of more expensive materials. Examples of such devices are those shown in the patents to Munroe, U.S. Pat. No. 2,217,917; Storm, U.S. Pat. No. 2,059,287; Chamberlin et. al., U.S. Pat. No. 2,784,722; and Jordan, U.S. Pat. No. 1,815,408.

Disposable flossers, on the other hand, have to be made inexpensively and the floss has to be permanently attached to the flosser in some way. Examples of patents disclosing disposable flossers are the Chodorow, U.S. Pat. No. 3,974,842; Katz, U.S. Pat. No. 3,926,201; and Henne, U.S. Pat. No. 2,187,899. Buscarino, U.S. Pat. No. 2,443,415 shows both disposable and permanent flossers. A major problem with disposable flossers of the prior art is the inadequate means of attachment of the floss, because in all cases the prior art requires either complex and expensive, or insufficient means of attachment. Thus, the Chodorow and Katz patents require that the flosser be molded about the floss, which is extremely expensive. Knotting has also been suggested, but this also is expensive and in some cases inadequate. Henne, U.S. Pat. No. 2,187,899, suggests various types of mechanical attachment, but none of these provide adequate anchoring; often when floss is forced between two adjacent teeth and the spacing is small, tremendous tensile force is applied and mechanical attachments of this type are not adequate, the floss ends merely pulling from their mechanical anchoring means.

With regard to the molding of the flosser about the ends of the floss, such as shown in the Chodorow and Katz patents, this produces not only a very expensive product, but also limits the types of floss which can be used. For example, medicated and flavored flosses, wherein the medicaments and flavorings are heat sensitive or volatile, cannot be used in the hot molding process because the heat of the operation and the molten plastic will degrade and/or drive off the medicament and/or flavoring material. Further, the medicaments and flavorings negatively impact the strength of the hot molded plastic as well as the polymer's ability to firmly affix the floss into the flosser device. See U.S. Patent Publication No. 2005/0205107 A1, and also the following U.S. Pat. Nos.:
U.S. Pat. Nos. 4,615,349, 3,974,842; 4,006,750; 4,016,892; 5,086,792; 5,538,023; 5,692,531; 3,858,594; 2,180,522; 2,187,899; 2,443,415; 3,926,201; 4,615,349; 413,001; 1,415,762; 2,702,555; 2,811,162; 3,693,594; 4,192,330; 4,522,216; 4,807,651; 5,113,885; and 5,123,432.

All of the foregoing dental flosser references are incorporated herein by reference.

Dental Picks:

In U.S. Pat. No. 4,651,760, a toothpick made from plastic is disclosed which has a pointed end supported by a mid portion having longitudinal notches which allows the toothpick to be compressed in a transverse direction and also has transverse flaps which may strike the tooth surfaces. A projecting plate is used as a handle. The flaps also insure that the toothpick attains considerable rigidity to keep its shape even though it may be subject to twisting.

In U.S. Pat. No. 4,805,646, a toothpick is disclosed which has a tapered triangular point which extends from a flexing joint which allows the point to assume various different angles. However, the tip itself is fairly rigid. See also U.S. Pat. Nos.:
U.S. Pat. Nos. 5,234,009; 1,527,028; 2,008,206; 3,101,172; 3,910,293; 4,135,528; 4,271,854; 4,314,574; 4,403,625; 4,570,653; 4,577,649; 4,942,034; 2,667,443; 2,748,781; 2,772,205; 2,896,639; 3,838,702; 3,897,795; 4,029,113; 4,175,326; 4,462,136; 4,510,127; and 4,627,975.

U.S. Pat. No. 4,616,667 discloses another toothpick design which has two ends which are pointed, having a shaft, a preferably substantially rectangular transverse cross-section and a longitudinally grooved cleaning tip which permits deformation of the tip in such a manner and direction to reach in between the narrow gaps of teeth while not sacrificing the longitudinal rigidity of the cleaning tip. Consequently, the area adjacent the tip may be bent but the tip itself remains rigid.

See the following relevant pick U.S. Pat. Nos.:
U.S. Pat. No. 5,234,009; 1,527,028; 2,008,206; 3,101,172; 3,910,293; 4,135,528; 4,271,854; 4,314,574; 4,403,625; 4,570,653; 4,577,649; 4,942,034; 2,667,443; 2,748,781; 2,772,205; 2,896,639; 3,838,702; 3,897,795; 4,029,113; 4,175,326; 4,462,136; 4,510,127; and 4,627,975.

All of the foregoing dental pick references are incorporated herein by reference.

Proxy Brushes:

Toothbrush designs have been suggested for gaining access to and for cleaning interproximal areas. Among the suggested designs are those disclosed in U.S. Pat. No. 3,939,520 to Axelsson and U.S. Pat. No. 3,720,975 to Nelson. In the Axelsson patent is disclosed a toothbrush with parallel bristles which extend in opposite directions from a central fin. Positioned at right angles to the fin is a flat base. The toothbrush is shaped so that, in use, the base of the fin is positioned on top of the gingival papilla and the bristles occupy the space above the base in the triangular portion of the interproximal space.

In the Nelson patent, a toothbrush is disclosed which has a conical shape provided by bristles which extend radially from a central core and which taper in length to form a tip. In the Nelson patent, the shape of the brush permits easy insertion of the brush between adjacent teeth. However, as in the Axelsson patent, the toothbrush in the Nelson patent causes the gingival papilla to be compressed.

Various teeth cleaning aids have been provided which perform the cleaning of ones teeth and massaging of the gums, the brushes taking on various forms such as wire bristles or foam material and generally are designed to rub or wipe perpendicularly across the gum and tooth surfaces. One brush which has been found useful in this manner is a small, twisted, wire-type, disposable brush wherein bristles are captured between and extend radially from a pair of twisted metal wires. Brushes of this type are shown and described in U.S. Pat. No. 4,780,923 issued to Schultheiss and U.S. Pat. No. 5,029,358 issued to Zimmerman. These devices have met with commercial success, however, due to their construction they are primarily limited for usage in the home as they are not simple to carry on the person of the user. It is therefore necessary to employ the use of toothpicks of wood, or other material, when it is desired to perform a cleaning function between the teeth when the user is at locations other than in the home. However, the use of such devices often is not as satisfactory as the employment of a brush of the type set forth above. See also U.S. Pat. Nos.:
U.S. Pat. Nos. 4,449,933; 5,327,377; 5,377,377; 1,770,195; 2,676,350; and 2,915,767.

All of the foregoing proxy brush references are incorporated herein by reference.

Dental Massagers:

Various elastomeric dental massagers, such as described in U.S. Pat. Nos. 1,069,874; 1,559,320; 1,608,212; 2,180,522; 2,788,000; 2,827,045; 2,981,204; 4,462,136; 5,704,388; and 6,283,751; can be used to stimulate blood flow in soft tissue.

All of the foregoing dental massager references are incorporated herein by reference.

Dental Irrigators:

In addition to the various interproximal devices described above, interproximal irrigates such as described in U.S. Pat. Nos. 3,468,306; 4,043,337; 4,265,229; 4,564,005; and 5,252,064 are useful for cleaning interproximal and subgingival spaces.

All of the foregoing dental irrigator references are incorporated herein by reference.

### Biofilms and Interproximal Devices

From 1960 thru 1982, numerous clinical studies reported that there is no clinical difference as to plaque removal and gingivitis scores between waxed and unwaxed multifilament dental floss. Note, both are “cord” flosses and contain sizing, binders, etc. These studies also confirmed that waxed and unwaxed floss are approximately 50% effective with respect to plaque removal and gingivitis scores. Thus the “cord” effect severely restricts efficiency of flossing and especially physical abrasive-type action associated with multifilament flosses that splay as described by Bass.

O'Leary in 1970, and Hill et al. in 1973, found no difference in the interproximal cleansing properties of waxed and unwaxed dental floss. This was reconfirmed in 1982 by Lobene et al., who showed no significant clinical difference on plaque and gingivitis scores. Similar results, i.e., no clinical difference between waxed and unwaxed multifilament dental floss with respect to reduced gingival inflammation were shown by Wunderlich in 1981. No differences in plaque removal were reported by Schmidt et al. in 1981 with multifilament flosses of various types. Stevens, 1980, studied multifilament dental floss with variable diameters and showed no difference in plaque and gingival health. Carter et al. 1975, studied professional and self administered waxed and unwaxed multifilament dental floss, both significantly, reduced gingival bleeding of interproximal and gingival sulci. Unwaxed multifilament dental floss appeared slightly, but not significantly more effective.

In view of this clinical work, it is not surprising that most of the multifilament dental floss sold today is, contrary to the teaching of Bass, bonded and/or waxed. The “bonding” in the yarn industry today is used more to facilitate processing and production during multifilament dental floss manufacture and packaging than for “flossing” reasons. Since clinical tests show no difference between waxed and unwaxed multifilament dental floss (both unfortunately are “bonded”), the multifilament dental floss industry has been comfortable with the yarn industry's propensity to use bonding agents in multifilament dental floss, thereby sacrificing splaying and physical abrasive-type cleaning. Of course, monofilament dental tapes do not splay and have a basic shortcoming with respect to abrasive-type cleaning.

The development of micromesh dental flosses with saliva soluble coatings and oriented HDPE dental tapes with saliva soluble coatings, which combine the strengths and advantages of multifilament dental flosses and monofilament dental tapes, while minimizing the shortcomings of monofilament and multifilament devices, are described in detail in copending: U.S. patent application, Ser. No. 10/073,682, entitled “Micromesh Interproximal Devices”, and U.S. patent application, Ser. No. 11/118,911, entitled “Coated Monofilament Oriented HDPE Dental Tape.” The saliva soluble coatings on these interproximal devices provide a means for delivering emulsions, flavors and sialagogues interproximally.

The classification of plaque as a biofilm is considered a major advance in the development of more effective “self-treatment” interproximal dental devices. See the following biofilm references:

Greenstein and Polson, J. Periodontol., May 1998, 69:5:507-520; van Winkelhoff, et al., J. Clin. Periodontol., 1989, 16:128-131; and Wilson, J. Med. Microbiol., 1996, 44:79-87.
Biofilms are defined as “ . . . matrix-enclosed bacterial population adherent to each other and to the surface or intersurfaces. These masses secrete an exopolysaccharide matrix for protection. Considerably higher concentrations of drugs are needed to kill bacteria in biofilms than organisms in aqueous suspensions.”

Costerton, J. W., Lewandowski, Z., DeBeer, D., Caldwell, D., Korber, D., James, G. Biofilms, the customized microniche. J. Bacterio., 1994, 176:2137-2142.
The unique attributes of biofilms are being recognized as increasingly important in the 1990's. Future studies into the mode of growth of biofilms will allow manipulation of the bacterial distribution.

Douglass, C. W., Fox, C. H. Cross-sectional studies in periodontal disease: Current status and implications for dental practice. Adv. Dent. Res., 1993, 7:26-31.

Greenstein, G. J., Periodontal response to mechanical non-surgical therapy: A review. Periodontol., 1992, 63:118-130.
Mechanical therapy remains effective with caveats to compliance and skill of therapists.

Marsh, P. D., Bradshaw, D. J. Physiological approaches to the control of oral biofilms. Adv. Dent. Res., 1997, 11:176-185.

Most laboratory and clinical findings support the concept of physiological control.

Further studies will reveal details of biofilm diversity.

Page, R. C., Offenbacher, S., Shroeder, H., Seymour, G. J., Kornman, K. S., Advances in the pathogenesis of periodontitis: Summary of developments, clinical implications and future directions. Periodont. 2000, 1997, 14:216-248.
Genetic susceptibility to three oral anaerobic bacteria play an important part in the progression of periodontitis. Acquired and environmental risk factors exacerbate the problem. Mechanical disruption will remain an effective and essential part of periodontal therapy. (emphasis added)

Papapanou, P. N., Engebretson, S. P., Lamster, I. B. Current and future approaches for diagnosis of periodontal disease. NY State Dent. J., 1999, 32-39.
New techniques are available such as a novel pocket depth measurement device, microscopic techniques, immunoassay, DNA probes, BANA hydrolysis tests. These more clearly define the nature of periodontitis.

The classification of plaque as a biofilm calls for more effective interproximal dental devices, with respect to removing, disrupting and/or controlling biofilms, increasing and prolonging saliva flow, and massaging soft tissue to enhance blood flow, which requires: (a) physical cleaning including particulate-abrasive-type cleaning interproximally and subgingivally when working the interproximal device between teeth, (b) topical coating of tooth surfaces including residual biofilms remaining after removing, disrupting and controlling biofilms, (c) increasing and prolonging saliva flow, and (d) delivering anti-inflammatories, while (e) enhancing blood flow of soft tissue through physical massaging of soft tissue with interproximal devices. Such physical-abrasive cleaning, combined with increased and prolonged saliva flow and delivering anti-inflammatory activity interproximally and subgingivally, is not available from commercial interproximal dental devices marketed today.

Xerostomia, the subjective feeling of oral dryness, is primarily caused by a marked decrease in the function of the salivary glands. Although not a disease, it may herald the onset, or signal the presence of a number of serious systemic diseases and conditions such as Sjogrens disease. Among these are the intake of xerogenic drugs, autoimmune diseases and radiation to the head and neck. Moreover, it may profoundly affect the soft and hard tissues of the mouth and interfere with alimentation and speech.

The dental ramifications of salivary gland hypofunction are quite well known. Saliva is a major protector of the tissues and organs of the oral cavity. In its absence, both the hard and soft tissues of the mouth may be severely damaged; the development of caries increases, the oral mucosa may become infected and/or ulcerated, and functions connected with the intake and digestion of foods may be impaired.

From an evolutionary viewpoint, the oldest function of the salivary glands has been to supply lubricatory molecules, not only to coat the food but also the soft and hard tissues. The lubricatory film allows food to travel easily through the digestive system, and provides smooth tissue surfaces with minimal function. Without appropriate lubrication, food is retained and impacted around teeth, making eating difficult and unpleasant and increasing plaque formation.

### SUMMARY OF THE INVENTION

The present invention discloses and claims various interproximal dental devices coated with: (a) a sialagogue, (b) a wax/sialagogue mixture, or (c) a saliva soluble coating containing a surfactant and a sialagogue, or (c) a saliva soluble emulsion/sialagogue mixture and associated methods for:
(a) controlling and/or disrupting and/or physically removing biofilms, while
(b) increasing and prolonging saliva flow.
   Preferably, the interproximal device is coated with:
(1) a sialagogue,
(2) a wax/sialagogue mixture,
(3) a surfactant/sialagogue mixture,
(4) a substantially water-free emulsion containing a sialagogue, or
(5) a saliva soluble coating comprising an emulsion having a surfactant continuous phase and polydimethylsiloxane discontinuous phase and at least one sialagogue.

The present invention also discloses and claims various biofilm-responsive interproximal dental devices treated with a sialagogue alone, a wax/sialagogue mixture, or a sialagogue in combination with a surfactant, or a sialagogue in combination with an emulsion, or a sialagogue in combination with an emulsion having a surfactant continuous phase and a polydimethylsiloxane discontinuous phase, all of which are suitable for increasing and prolonging saliva flow. In a preferred embodiment, the device of the invention also delivers anti-inflammatory effects in those areas of the oral cavity reached by said interproximal device.

### PREFERRED EMBODIMENTS

One embodiment of the present invention further discloses and claims various treatments for xerostomia including impaired salivary function in patients with noninsulin-dependent diabetes mellitus.

Accordingly, one embodiment of the present invention comprises biofilm-responsive, interproximal dental devices suitable for controlling, disrupting and removing biofilms, while increasing and prolonging saliva flow, and imparting anti-inflammatory activity interproximally and subgingivally.

A further embodiment of the present invention comprises saliva soluble coated interproximal dental devices containing a releasable sialagogue with particulate soft abrasives imbedded in the coating, thereby rendering the interproximal dental device biofilm-responsive and suitable for increasing and prolonging saliva flow and anti-inflammatory activity.

Another embodiment of the invention comprises a self-treatment means comprising a sialagogue treated interproximal dental device suitable for routinely removing and disrupting biofilms formed on interproximal, supragingival and subgingival tooth surfaces, and for coating residual biofilms that remain interproximally after use of the interproximal device with sialagogue, while also increasing, and prolonging saliva flow and anti-inflammatory activity interproximally and subgingivally, thereby maintaining control of biofilms.

Still another embodiment of the invention comprises a method for overcoating saliva soluble, coated, dental devices with imbedded particulate abrasives of various particle sizes and particle size distributions as a means for effectively removing and disrupting biofilms and stains from interproximal tooth surfaces, while simultaneously treating residual biofilm with a biofilm-responsive dental device containing sialagogues, increasing and prolonging saliva flow, and introducing anti-inflammatory activity interproximally and subgingivally.

Yet another embodiment of the invention comprises a patient self-treatment method for periodically (i.e., hourly, daily, weekly, monthly—as needed) removing and disrupting biofilms that form on interproximal, supragingival and subgingival tooth surfaces, and treating residual biofilms with a sialagogue or a sialagogue-containing coating that helps control biofilms, while simultaneously increasing, prolonging saliva flow, and introducing anti-inflammatory activity interproximally and subgingivally.

A further embodiment of the invention comprises biofilm-responsive, interproximal dental devices overcoated with imbedded particulate abrasives and containing a releasable saliva soluble base coating which contains a sialagogue suitable for increasing and prolonging saliva flow and introducing anti-inflammatory activity interproximally and subgingivally, while simultaneously controlling, disrupting and removing biofilms from interproximal tooth surfaces.

Another embodiment of the invention comprises biofilm-responsive, interproximal dental devices containing a sialagogue, overcoated with imbedded particulate soft abrasives suitable for controlling, disrupting and removing biofilms, while delivering dry mouth and anti-inflammatory relief interproximally and subgingivally among at-risk adults.

Still another embodiment of the invention comprises biofilm-responsive, interproximal dental devices containing a sialagogue, overcoated with soft abrasives suitable for controlling, disrupting and removing biofilms, for treating dry mouth, while increasing and prolonging saliva flow and for treating inflammation, where the soft abrasives include: silica, pumice, alumina, calcium carbonate and/or dicalcium phosphate dihydrate.

Yet another embodiment of the invention comprises biofilm-responsive, interproximal dental devices treated with a sialagogue suitable for controlling, disrupting and removing biofilms, while increasing and prolonging saliva flow and introducing anti-inflammatory activity interproximally and subgingivally; provided with an overcoating of imbedded, particulate, soft abrasives, where said abrasives contain other substances ranging from flavorants, antimicrobials and cleaning substances to mouth conditioners and various pharmaceutical substances.

Another embodiment of the invention comprises improved interproximal dental devices suitable for controlling, disrupting and removing biofilms, while increasing saliva flow and introducing anti-inflammatory activity interproximally and subgingivally, with an overcoating of imbedded, particulate, soft abrasives containing a saliva soluble substance with at least one sialagogue and preferably mouth conditioners, tartar control agents, etc.

Yet another embodiment of the invention comprises a method for improving interproximal dental devices with saliva soluble coatings containing at least one sialagogue suitable for increasing and prolonging saliva flow and introducing anti-inflammatory activity interproximally and subgingivally, comprising sequential overcoating of said saliva soluble base coated device with two or more particulates having substantially different densities, wherein said various particulates are imbedded into said base coating prior to cooling and solidifying.

Still another embodiment of the invention comprises improved commercial, emulsion coated, interproximal dental devices containing a sialagogue with an overcoating of imbedded, particulate, soft abrasive suitable for controlling, disrupting and removing biofilms, while simultaneously increasing and prolonging saliva flow and introducing anti-inflammatory activity interproximally and subgingivally.

Still another embodiment of the invention comprises sialagogue-treated interproximal dental devices and associated methods for: (a) removing, disrupting and controlling interproximally and subgingivally, the supragingival and subgingival microbiological burden associated with biofilms, (b) increasing and prolonging saliva flow, and (c) introducing anti-inflammatory activity; interproximally and subgingivally to at-risk adults suffering from dry mouth.

The present invention additionally discloses and claims various methods for manufacturing various interproximal dental devices treated with sialagogues or various coatings containing one or more sialagogues.

Another embodiment discloses and claims various methods for employing sialagogue-treated dental devices in controlling biofilms interproximally, for simultaneously controlling saliva flow, while imparting anti-inflammatory properties interproximally and subgingivally.

### DEFINITIONS

For purposes of describing the present invention, the following terms are defined as set out below:

As used herein, the term “sialagogue(s)” is defined as a natural or synthetic compound or mixture of compounds that cause an increase in saliva in the mouth. In other words, sialagogues are substances that stimulate the production of saliva.

There are two important types of sialagogues:

(A) Gustatory sialagogues, i.e., materials related to the sense of taste, such as particular foods and flavors. Particularly preferred sialagogues include: ascorbic acid, black pepper, ginger, licorice, pilocarpine, affinin, spilanthol, bethanechol chloride, cayenne pepper, echinacea, verba santa, bay berry, sanguinarine, ginseng, kava, kudzu, capsaicin, zingerone, eugenol, and piperine.

(B) Pharmaceutical sialagogues (also called parasympathomimetic agents) which improve salivation.

Examples of other preferred sialagogues of both types, useful in the present invention, are the following:
1. Plant extracts including: amides of vegetable origin including:
   (a) affinin, i.e. N-isobutyl-2,6,8-decatrienamide,
   (b) Ciluan Root derivatives, including Heliopsis Longipes,
   (c) bioactives N-isobutylamides from buds of Spilanthes acmella,
   (d) alkamides present in flavoring plants including affinin and capsaicin, and
   (e) N-alkyl-carboxamide compounds, including 3-(1-menthoxy) propane-1,2 diol 1(2-hydroxyphenyl)-4-(3-nitrophenyl)-1,2,3,5-tetrahydropyrimidine-2-one; and
2. compositions such as described in U.S. Pat. Nos. 5,585,424; 6,780,443; 6,890,567; 6,899,901; U.S. Patent Publication No. 2003/0215532 and U.S. Patent Publication No. 2004/0052735. See also: Journal of the Society of Cosmetic Chemists, 29:185-200 (1988) H. R. Watson. Preferably such compositions contain at least one Jambu Oleoresin and one Spilanthol;
3. “Spilanthes” which are a strong anti-bacterial herb with in-vitro activity against such common pathogens as: Escherichia coli, Klebsiella pneumoniae, Proteus vulgaris, Pseudomonas aeruginosa, Salmonella gallinarum and Staphylococcus albus. Spilanthes also inhibits the yeast/fungal organism Candida albicans, which is responsible for the nearly epidemic condition known as candidiasis (thrush);
4. “Heliopsis longipes”, which are herbaceous plant species found in Mexico, have long been used there primarily as a spice or flavoring, as chewing the root causes numbness and tingling in the mouth and tongue and stimulates salivation. Little, Jr., E. L., Journal of the Washington Academy of Sciences, Vol. 38, No. 8, pp. 269-274 (Aug. 15, 1948). These roots were also chewed to relieve toothache. Id.
5. Extracts from the roots of Heliopsis longipes have been used in a few medical applications. Id. For example, reportedly, such an extract has been used for treating colds and pneumonia, and an alcoholic extract has been tested for use as an anesthetic for tooth extraction. Id. It has also been reported that an extract of these roots possesses antiseptic properties. Molina-Torres, J., et. al. Antimicrobial properties of alkanides present in flavouring plants traditionally used in Mesoamerica: affinin and capsaicin, Journal of Ethnopharmacology, Vol. 64, Iss. 3, pp. 241-248 (March 1999). A crude methanol extract of Heliopsis longipes roots has been described as having the potential to generate anti-infective agents, although this extract reportedly does not show any activity in plate diffusion tests against either E. coli (Gram negative bacteria) or B. subtilis (Gram positive bacteria). Id.; and Gutierrez-Lugo, M. T., et. al., Antimicrobial and cytotoxic activities of some crude drug extracts from Mexican Medicinal plants, phyomedicine, Vol. 2 (4), pp. 341-347 (1996). An ethanol extract of Heliopsis longipes roots has been reported as having variable bactericidal effects on E. coli and S. aureus, Romero-R., C. M., et. al., Preliminary Studies of the Antibacterial, Insecticidal, and Toxicological Effects of Chiluan Root (Heliopsis Longipes), as translated, Veterinaria Mexico, pp. 151-156 (1989).
6. Heliopsis longipes roots are known to contain a bioactive alkamide, affinin, identified as N-isobutyl-2E, 6Z, 8E-decatrienamide or N-isobutyldeca-trans-2,cis-6,-trans-8-trienamide. Respectively, Id.; and Crombie, L., et. al., Amides of Vegetable Origin, part X. The Stereochemistry and Synthesis of Affinin, Journal of Chemical Society, pp. 4970-4976 (1963).
7. Affinin has also been identified as N-isobutyl 2,6,8-decatrienoamide in one publication, in another publication, and N-isobutyldodeca-2-trans-6-cis-8-trans-trienamide in another publication. Respectively, Jacobson, M., et. al., Correction of the Source of “Affinin” (N-Isobutyl-2,6,8-Decatrienoamide, Journal of Organic Chemistry 12, pp. 731-732 (1947) (emphasis added); and Ogura, M., et. al., Ethnopharmacologic studies. I. Rapid solution to a problem—oral use of Heliopsis longipes—by means of multidisciplinary approach, Journal of Ethnopharmacology, 5, pp. 215-219 (1982) (emphasis added). Purified affinin, prepared from an ethanol extract of Heliopsis longipes roots, has been reported as being toxic to certain microorganisms, the toxicity varying for Gram positive and Gram negative bacteria. Molina-Torres, J., et. al.
8. An aqueous solution of affinin, prepared from a powder of an ethanol extract of Heliopsis longipes roots, has also been reported as having an analgesic effect when administered orally to mice at doses from 2.5 to 10.0 mg/kd, with severe depression of normal motor activity and two out of five deaths occurring at the highest dose. Ogura, M., et. al.
   In the one publication where affinin is identified as N-isobutyl-dodeca-2-trans-6-cis-8-trans-trienamide, it was said to be identical with spilanthol, the pungent principle of several Spilanthes species. Ogura, M., et. al.
   However, in the publication of Little, Jr., affinin is said to be similar to spilanthol, which has been isolated from flower heads of a species of Spilanthes, Little, Jr., E. L., at p. 270. The flowers and leaves of Spilanthes acmella L. var. oleracea Clarke are reported as having been used as a spice and as a folk medicine for stammering, toothache, stomatitis and throat complaints. Ramsewak, R. S. et. al., Bioactive N-isobutylamides from the flower buds of Spilanthes acmella, Phytochemistry 51, pp. 729-732 (1999).
   The Heliopsis longipes extract may be prepared using standard means or methods, such as by contacting the plant material with an appropriate solvent to prepare a botanical tincture, or by any other conventional means or method, such as by CO₂ extraction, freeze-drying, spray-drying, and the like. (See Gennaro A R; Remington: The-Science and Practice of Pharmacy, Mack Publishing Company, Easton Pa. 1995 and The United States Pharmacopeia 22^{nd} Rev, and The National Formulary (NF) 17 ed, USP Convention, Rockville, Md., 1990.) The extract is prepared using a root or roots of Heliopsis longipes and a solvent, such as water combined with other solvents, an organic solvent, such as hexane and glycerin, or an alcohol, such as ethanol, or any combination thereof. Preferably, an alcohol or a hydro-alcohol solvent is used, and most preferably, ethanol or a combination of ethanol and water is used.
   The resulting extract is typically composed of a wet or liquid component that is light brown to golden in color and a dry or solid component, in amounts of about 90.0 to about 99.9 weight percent, such as about 98 weight percent, and about 10 to about 0.01 weight percent, such as about 2 weight percent, respectively, relative to the extract. The composition, including the extract in the wet-dry form just described, may be formulated as a powder or paste, such as a powder including about 66.6 weight percent extract and 33.4 weight percent carrier on a wet basis; or about 0.01 to about 100 weight percent extract on a dry basis-including the natural product sprayed on itself, such as about 2 to 10 weight percent extract on a dry basis, or in any combination or permutation for either method-wet or dry. All of the foregoing references regarding sialagogues are incorporated herein by reference.

“Interproximal dental devices” are defined as medical devices treated with a sialagogue, or having a saliva soluble coating containing a sialagogue, which devices are suitable for physically controlling, disrupting and removing biofilms, while releasing sialagogues or saliva soluble coatings containing sialagogues that control and disrupt biofilms, while simultaneously increasing and prolonging saliva flow, and introducing anti-inflammatory activity interproximally. Interproximal dental devices include: dental floss, dental tape, dental flossers, proxy brushes, dental stimulators, dental massagers, dental picks, interproximal irrigators, etc., including:
1. Multifilament dental flosses such as described in U.S. Pat. Nos.:
U.S. Pat. Nos. 4,911,927; 4,029,113; 4,610,872; 4,034,771; 5,908,039; 2,667,443; 3,830,246; 1,149,376; 1,069,874; 5,830,495; 2,748,781; 1,138,479; 1,839,486; 1,943,856; 6,080,481; 2,700,636; 3,699,979; 3,744,499; 3,837,351; 4,414,990; 3,330,732; 5,967,155; 5,937,874; 5,505,216; 5,503,842; 5,032,387; 4,950,479; 5,098,711; 1,989,895; 5,033,488; 2,542,518; 2,554,464; 1,285,988; 1,839,483; 4,151,851; 2,224,489; 2,464,755; 2,381,142; 3,800,812; 3,830,246; 3,897,795; 3,897,796; 4,215,478; 4,033,365; 3,771,536; 3,943,949; 6,016,816; 6,026,829; 5,353,820; 5,557,900; 5,226,435; 5,573,850; 5,560,377; 5,526,831; 5,423,337; 5,220,932; 4,548,219; 3,838,702; 5,904,152; 4,911,927; 5,165,913; and 5,098,711;
   The multifilament dental floss of the present invention may be more appropriately described as a local delivery system, suitable for the topical cleaning and/or treatment of those hard-to-reach areas between teeth (interproximal spaces) and the areas immediately below the gumline (i.e., the gingival crevice).
   The unique construction of multifilament dental floss and loading of cleaners, active ingredients and conditioners in the space around each of the nylon fibers allows loading of these substances from 10 to over 100% by weight of the floss. The “loaded” floss tends to “splay” (open up), when pressure is applied to the floss, i.e., the pressure required to fit the floss between teeth and/or, after the floss is positioned between teeth, the pressure applied during flossing. When the floss is splayed, the loaded substances are release and continue to be released during the sawing motion of flossing. This releasing action supplements the cleaning action of flossing by releasing cleaners to work with the floss.

In a preferred embodiment, the present invention may be defined as the treatment of the oral cavity with an interproximal delivery system comprising:
a. floss having from between 2 and 12 strands, each containing between about 100 and 800 filaments with a denier between 300 and about 1200, and
b. a cleaning preparation containing a sialagogue at from between 5 and about 100% by weight of said strands, wherein:
   i. said filaments are substantially free from sizing and binding agents;
   ii. said interproximal delivery system splays upon being worked between interproximal surfaces;
   iii. said interproximal delivery system release from between about 10 and about 80% by weight of said cleaning preparation upon splaying; and
   iv. said cleaning preparation:
      (a) is loaded into said delivery system as a substantially aqueous free, hot-melt emulsion,
      (b) is contained throughout the interproximal delivery system, primarily in the area surrounding said filaments with less than about 5% by weight of said cleaning preparation on the outermost surface of said delivery system,
      (c) can contain up to about 50% by weight of an active chemotherapeutic agent selected from the group consisting of: antimicrobials, antibiotics, antioxidants, desensitizers and anti-tartar agents, and
      (d) contains at least one sialagogue.
   2. Texturized multifilament dental flosses such as described in U.S. Pat. No. 5,711,935.
      Virtually all natural and man-made fibers and specifically filament yarns, twisted continuous filament yarns and the like can be texturized, including: nylon, polyester, polypropylene and various blends of such fibers and/or filaments. Texturizing can be achieved with false twist, core-bulked, textralized, edge crimping, gear crimped, air jet, crinkle, cotexturizing, etc. type processes or modifications thereof as described in E. Stacy. Hosiery Res. Bull. (1): 1-22, 1957 and the references cited at pp. 20-22 of Textured Yarn Technology, Vol. 1. Monsanto, 1967. See also Textured Yarns. Monsanto, 1962 and Stretch Woven Fabrics and Textured Yarns, Monsanto 1963.
      These “bulling” processes can be carried out with various types of texturizing equipment as described at pp. 223-233 of Textured Yarn Technology, Vol. 1, Monsanto, 1967, including for instance: Hobnourn No. 2086, ARCT: FT-V, FT-1, FT-F, FT-3, FT-400, FT-U and FT-415; Carolina UNRAV Purdy-Hodges Yarn Crimper, Whitlin RD-5; U.S. Acme Jumbo & Model I Texturizers; Fujiloft F205 Heberlein High Performance Universal Texturizing Machine, Autotwister TZ21, False Twister F231, Ratti Model TRS, Berliner SDD-Set 280, Two-For-One Twister; Turbo-Twist, Pinion & Turbo-Duo Twist.
      The equipment and processes used for texturizing are described in numerous U.S. patents and literature as set forth below, the disclosures of which are hereby incorporated by reference into this specification: U.S. Pat. Nos. 2,463,620; 2,655,781; 2,463,618; 2,904,952; 2,803,105; 23,803,108; and 2,803,109.
      “A survey of the literature and patents relating to Bulked Continuous-Filament Yarns”, E. Hutton and W. J. Morris, The Cotton Silk and Man-Made Fibres Research Assoc., Shirley Inst. Didsbury, Manchester, England (1963).
      “Bulk, Stretch and Texture”, The Textile Inst., Manchester England 1966 and the various references cited at pp. 97-104 of Textured Yarn Technology, Vol. 1; Monsanto, 1967.
      See also: U.S. Pat. Nos. 2,790,611; 2,919,534; 2,921,358; 2,925,641; 2,931,089; 2,977,661; 2,977,746; 2,977,663; 3,017,684; 3,021,588; 3,025,584; 3,028,653; 3,028,654; 3,035,328; and 3,047,932; British Patent Nos. 772,756; 761,084; 801,147; 802,689; and 839,393.
      See also yarns and equipment described at pp. 204-234 in Texturized Yarn Technology, Monsanto, Vol. 1 1967, and U.S. Pat. Nos. Re. 23,824; 2,575,837; 2,505,618; 2,514,557; 2,575,781; 2,575,833; 2,575,837; 2,575,838; 2,575,839; 2,647,285; 2,734,228; 2,734,229; 2,734,251; 2,734,252; 2,740,992; 2,758,357; 2,758,358; 2,760,252; 2,854,701; 2,854,728; 2,854,729; 2,960,729; 2,960,730; 2,997,747; 3,000,059; 3,000,060; 3,016,677; 3,027,108; 2,783,609; 2,852,906; 2,868,967; 2,958,112; 2,994,938; 3.013,379; 3,017,737; 3,043,088; and 3,091,908.
   3. Monofilament dental tapes including:
      (a) PTFE dental tapes as described in U.S. Pat. Nos.:
U.S. Pat. Nos. Re. 35,439; 3,800,812; 4,974,615; 5,760,117; 5,433,226; 5,479,952; 5,503,842; 5,755,243; 5,845,652; 5,884,639; 5,918,609; 5,962,572; 5,998,431; 6,003,525; 6,083,208; 6,198,830; 6,161,555; 6,027,192; 5,209,251; 5,033,488; 5,518,012; 5,911,228; 5,220,932; 4,776,358; 5,718,251; 5,848,600; 5,787,758; 5,765,576; 3,664,915; 3,953,566; 3,962,153; 4,096,227; 4,187,390; 4,256,806; 4,385,093; 4,478,665; 4,776,358; 5,518,012 and 5,765,576;
   (b) Fibaclean®—type dental tapes, such as described in U.S. Pat. No. 6,591,844;
   (c) micromesh dental tapes as described in U.S. patent application, Ser. No. 10/331,800; and
   (d) oriented high density dental tapes as described in U.S. patent application, Ser. No. 11/118,911;
   4. Other suitable interproximal devices include dental flossers, dental picks (stimulators), proxy brushes and interdental irrigators, which are discussed in detail below.
      (a) Dental Flossers:
         For more than one hundred years there have been attempts to improve and modernize the dental flossing technique by development of holders onto which floss is detachably or permanently secured, and a few hundred patents have already been granted on various flossing devices. These go back to the Shurtleft, U.S. Pat. No. 147,987 of 1874, and Wallace, U.S. Pat. No. 175,795 of 1876. In general, the dental flossers of the prior art have fallen into two basic categories, namely the reusable or permanent dental flossers wherein the customer threads the device with floss and then throws away the floss after use, but retains the flosser; and the disposable dental flossers having a structure to which the floss is permanently attached, the entire device being thrown away after one or more uses and replaced by another similar device.
         In general, the dental flossers of the permanent type are, as would naturally be expected, of considerably more complex construction and made of more expensive materials. Examples of such devices are those shown in the patents to Munroe, U.S. Pat. No. 2,217,917; Storm, U.S. Pat. No. 2,059,287; Chamberlin et. al., U.S. Pat. No. 2,784,722; and Jordan, U.S. Pat. No. 1,815,408.
         Disposable flossers, on the other hand, have to be made inexpensively and the floss has to be permanently attached to the flosser in some way. Examples of patents disclosing disposable flossers are; Chodorow, U.S. Pat. No. 3,974,842; Katz, U.S. Pat. No. 3,926,201; and Henne, U.S. Pat. No. 2,187,899. Buscarino, U.S. Pat. No. 2,443,415, shows both disposable and permanent flossers. A major problem with disposable flossers of the prior art is the inadequate means of attachment of the floss, because in all cases the prior art requires either complex and expensive, or insufficient means of attachment. Thus, the Chodorow and Katz patents require that the flosser be molded about the floss, which is extremely expensive. Knotting has also been suggested, but this also is expensive and in some cases inadequate. Henne, U.S. Pat. No. 2,187,899, suggests various types of mechanical attachment, but none of these provide adequate anchoring; often when floss is forced between two adjacent teeth and the spacing is small, tremendous tensile force is applied and mechanical attachments of this type are not adequate, the floss ends merely pulling from their mechanical anchoring means.
         With regard to the molding of the flosser about the ends of the floss, such as shown in the Chodorow and Katz patents, this produces not only a very expensive product, but also limits the types of floss which can be used. For example, medicated and flavored flosses, wherein the medicaments and flavorings are heat sensitive or volatile, cannot be used in the hot molding process because the heat of the operation and the molten plastic will degrade and/or drive off the medicament and/or flavoring material. Further, the medicaments and flavorings negatively impact the strength of the hot molded plastic as well as the polymer's ability to firmly affix the floss into the flosser device. See U.S. Patent Publication No. 2005/0205107 Al and also the following U.S. Pat. Nos.:
U.S. Pat. Nos, 4,615,349, 3,974,842; 4,006,750; 4,016,892; 5,086,792; 5,538,023; 5,692,531; 3,858,594; 2,180,522; 2,187,899; 2,443,415; 3,926,201; 4,615,349; 413,001; 1,415,762; 2,702,555; 2,811,162; 3,693,594; 4,192,330; 4,522,216; 4,807,651; 5,113,885; and 5,123,432.
   All of the foregoing dental flosser references are incorporated herein by reference.
   (b) Dental Picks:
      In U.S. Pat. No. 4,651,760, a toothpick made from plastic is disclosed which has a pointed end supported by a mid portion having longitudinal notches which allows the toothpick to be compressed in a transverse direction and also has transverse flaps which may strike the tooth surfaces. A projecting plate is used as a handle. The flaps also insure that the toothpick attains considerable rigidity to keep its shape even though it may be subject to twisting.
      In U.S. Pat. No. 4,805,646, a toothpick is disclosed which has a tapered triangular point which extends from a flexing joint which allows the point to assume various different angles. However, the tip itself if fairly rigid.
      U.S. Pat. No. 4,616,667 discloses another toothpick design which has two ends which are pointed, having a shaft, a preferably substantially rectangular transverse cross-section and a longitudinally grooved cleaning tip which permits deformation of the tip in such a manner and direction to reach in between the narrow gaps of teeth while not sacrificing the longitudinal rigidity of the cleaning tip. Consequently, the area adjacent the tip may be bent but the tip itself remains rigid.
         See also U.S. Pat. Nos.:
U.S. Pat. Nos. 5,234,009; 1,527,028; 2,008,206; 3,101,172; 3,910,293; 4,135,528; 4,271,854; 4,314,574; 4,403,625; 4,570,653; 4,577,649; 4,942,034; 2,667,443; 2,748,781; 2,772,205; 2,896,639; 3,838,702; 3,897,795; 4,029,113; 4,175,326; 4,462,136; 4,510,127; and 4,627,975.
   All of the foregoing dental pick references are incorporated herein by reference.
   (c) Proxy Brushes:
      Toothbrush designs have been suggested for gaining access to and for cleaning interproximal areas. Among the suggested designs are those disclosed in U.S. Pat. No. 3,939,520 to Axelsson and U.S. Pat. No. 3,720,975 to Nelson. In the Axelsson patent is disclosed a toothbrush with parallel bristles which extend in opposite directions from a central fin. Positioned at right angles to the fin is a flat base. The toothbrush is shaped so that, in use, the base of the fin is positioned on top of the gingival papilla and the bristles occupy the space above the base in the triangular portion of the interproximal space.
      In the Nelson patent, a toothbrush is disclosed which has a conical shape provided by bristles which extend radially from a central core and which taper in length to form a tip. In the Nelson patent, the shape of the brush permits easy insertion of the brush between adjacent teeth. However, as in the Axelsson patent, the toothbrush in the Nelson patent causes the gingival papilla to be compressed.
      Various teeth cleaning aids have been provided which perform the cleaning of ones teeth and massaging of the gums, the brushes taking on various forms such as wire bristles or foam material and generally are designed to rub or wipe perpendicularly across the gum and tooth surfaces. One brush which has been found useful in this manner is a small, twisted, wire-type, disposable brush wherein bristles are captured between and extend radially from a pair of twisted metal wires. Brushes of this type are shown and described in U.S. Pat. No. 4,780,923 issued to Schultheiss and U.S. Pat. No. 5,029,358 issued to Zimmerman. These devices have met with some commercial success, however, due to their construction they are primarily limited for usage in the home as they are not simple to carry on the person of the user. It is therefore necessary to employ the use of toothpicks of wood, or other material, when it is desired to perform a cleaning function between the teeth when the user is at locations other than in the home. However, the use of such devices often is not as satisfactory as the employment of a brush of the type set forth above.
         See also U.S. Pat. Nos.:
U.S. Pat. Nos. 4,449,933; 5,327,377; 5,377,377; 1,770,195; 2,676,350; and 2,915,767.
   All of the foregoing proxy brush references are incorporated herein by reference.
   (d) Dental Massagers:
   As used herein, the term “dental massagers” includes dental devices described in U.S. patents: U.S. Pat. Nos. 1,069,874; 1,559,320; 1,608,212; 2,180,522; 2,788,000; 2,827,045; 2,981,204; 4,462,136; 5,704,388; and 6,283,751; can be used to stimulate blood flow in soft tissue.
      All of the foregoing dental massager references are incorporated herein by reference.
   (e) Dental Irrigators:
      As used herein, the term “dental irrigators” includes various dental devices suitable for introducing liquids between teeth and subgingivally under pressure, such as those described in the following U.S. Pat. Nos. 3,468,306; 4,043,337; 4,265,229; 4,564,005; 5,252,064. The various liquids used in these devices can contain sialagogues, sialagogue/surfactants and sialagogue/emulsion combinations, as detailed herein.
      All of the foregoing dental irrigator references are incorporated herein by reference.

The terms “fiber” and “filament” are used synonymously throughout this specification in a manner consistent with the first three definitions of “fiber” and the first definition of “filament” as given in the New Illustrated Webster's Dictionary, ©1992 by J.G. Ferguson Publishing Co. the relevant disclosure of which is hereby incorporated herein by reference.

“Dry mouth, at-risk patients” defines those patients suffering from reduced saliva flow, such as those who have one or more chronic diseases which they regularly treat with medicine having an adverse side reaction that causes dry mouth and/or patients who receive treatments that adversely affect saliva flow such as radiation therapy and chemotherapy that typically cause dry mouth. See also:

(a) McDonald E. and Marino C. “Dry Mouth: Diagnosing and treating its multiple causes.” Geriaticis 1991; 46 (Mar. 61-63);

(b) Sreebyn L. M. and Valdini A. “Xerostomia Part 1: Relationship to other oral symptoms and salivary gland hypofunction.” Oral Surg. Oral Med. Oral Pathol. 1988, 66:451-8;

(c) Sreebyn L. M. “Salivary flow in health and disease.” Compend. Contin. Educ. Dent. Suppl. No. 13:461-469;

(d) Mandel I.W. “The role of saliva in maintaining oral homeostasis.” JADA Vol. 119, Aug. 1989; 298-304; and

(e) Mandel I. W. “The functions of saliva.” J. Dent. Res. 66 (Spec Iss) 623-627; February 1987.

All of the foregoing are incorporated herein by reference.

“Saliva flow” is defined as an important element in oral hygiene. Saliva flow tends to wash the mouth of food and contaminants, promotes a balanced ecology of the oral cavity including the gums and teeth, and refreshes the mouth. Stimulating saliva flow alleviates xerostomia and the sensations and perception of dry mouth; reduces oral bacteria, dental caries, halitosis, gingivitis, periodontitis, and oral plaque (biofilms); and promotes the healing of and alleviation of oral lesions, such as lesions present in the mouth, including lesions induced by stomatitis, herpes, and the like.

“Wax/sialagogue mixtures” are defined to include; (a) various water-soluble waxes such as PEG, alone or with a flavor solubilizing surfactant, (b) Lipowax (mixtures of fatty acid PEG surfactant, a long chain alcohol), and (c) emulsifying waxes.

“Saliva soluble coatings” for the various interproximal dental devices of the invention are defined as those saliva soluble substances that coat interproximal dental devices for purposes of: lubrication and ease of device insertion interproximally and for delivering interproximally, cleaners, therapeutic antimicrobials, flavors, sialagogues and other additives. These saliva soluble coatings generally comprise from between about 25 to about 100% by weight of the interproximal dental device substrate.

Preferred saliva soluble, base coatings which can contain sialagogues include:
(a) those emulsion coatings described in the following U.S. Pat. Nos. 4,950,479; 5,032,387; 5,538,667; 5,561,959; and 5,665,374, which are hereby incorporated by reference,
(b) various dental device coatings, such as described in U.S. patents: U.S. Pat. Nos. 5,908,039; 6,080,495; 4,029;113; 2,667,443; 3,943,949; 6,026,829; 5,967,155 and 5,967,153, which are hereby incorporated by reference,
(c) those substantive saliva soluble coatings described and claimed in U.S. Pat. Nos. 6,907,889; 6,609,527; 6,916,880 and 6,545,077, which are hereby incorporated by reference, and
(d) those coatings described in copending applications: U.S. Ser. No. 11/096,606, filed Apr. 1, 2005, entitled: “Coated Monofilament Tape Bobbins and Methods for Winding” published as U.S. Patent Publication No. 2005-0199334 A1; U.S. Ser. No. 11/149,597, filed Jun. 10, 2005, entitled: “Non-Crystalline Saliva-Soluble Coatings for Elastomeric Monofilament Dental Tapes” published as U.S. Patent Publication No. 2005-0226820 A1; and U.S. Ser. No. 11/196,827, filed Aug. 3, 2005, entitled: “Biofilm Therapy Interproximal Devices.”

All of the foregoing coatings can contain biofilm-responsive levels of one or more substances suitable for controlling and disrupting biofilms and at least one sialagogue for prolonging and increasing saliva flow and controlling anti-inflammatory activity interproximally and subgingivally.

As used herein, “coating” is generally defined as the process of introducing oral care substances onto various interproximal device substrates and includes: waxing, compression coating, contact coating, dipping, spraying, soaking, etc.

As used herein, the phrase “wax coating” refers to the coating process generally used for most multifilament dental devices and is described in U.S. Pat. Nos. 2,667,443; 5,830,495; 5,908,039 and 5,967,153 and the references cited therein, which are hereby incorporated by reference. Wax coatings are comprised primarily of a suitable wax, such as microcrystalline wax, paraffin wax, beeswax, PEG, and the like, into which is admixed small amounts of flavors, sialagogues, sweeteners, and other low-level active ingredients commonly used in dental floss manufacture. As the wax coating is worked across tooth surfaces during flossing, the wax may be partially removed from the floss substrate and deposited onto tooth surfaces. Generally, very little of the minor ingredients mixed into the wax are released into the oral cavity due to the hydrophobic barrier to saliva solubility indicated by the wax.

As used herein, the phrase “compression coating” refers to the coating process used for both multifilament and monofilament devices as described in U.S. Pat. Nos. 4,911,927; 5,098,711; 5,165,913; 5,651,959; 5,665,374; 5,711,935; 6,545,077; 6,575,176; 6,591,844; 6,604,534 and 6,609,527, which are hereby incorporated by reference. Commercial dental devices coated by compression coating include: REACH® Gentle Gum Care, easySLIDE Pro™, and CleanPaste™.

As used herein, the phrase “contact coating” describes as the preferred means for coating various “thin” monofilament interproximal devices, where the coating substance is generally a liquid, low melt viscosity mixture or emulsion which is transferred onto both sides of the monofilament substrate as it is passed over various contact loading means generally described in U.S. Pat. Nos. 2,667,443; 5,830,495; 5,908,039 and 5,967,153.

As used herein, the phrase “contact coatings” generally describes various oral care containing substances suitable for contact coating various monofilament interproximal tapes. These contact coatings include various low melt viscosity mixtures and emulsions.

As used herein, the phrase “low melt viscosity mixtures and emulsions” generally describes those low viscosity liquid oral care substances containing at least one sialagogue including mixtures, emulsions, water soluble coatings suitable for coating onto various monofilament interproximal devices using various coating means including waxing, contact coating, compression coating and the like.

As used herein, the terms “MICRODENT®” and “ULTRAMULSION®” refer to emulsions of polydimethylsiloxane at various molecular weights in various poloxamer surfactants as described and claimed in U.S. Pat. Nos. 4,911,927; 4,950,479; 5,032,387; 5,098,711; 5,165,913; 5,538,667; 5,645,841; 5,651,959 and 5,665,374. These mouth conditioners are preferably included in crystal-free contact coatings of various interproximal dental devices of the invention. It has been discovered that sialagogues can be mixed with these emulsions as described herein.

As used herein, the phrase “saliva-soluble, crystal-free coatings” refers to those sialagogue-containing coating emulsions that indicate substantial flake resistance, yet release from various interproximal dental devices of the present invention during use when exposed to saliva in the oral cavity. These coatings can include SOFT ABRASIVES® that are dispersed and not solubilized in said coatings. These SOFT ABRASIVES® remain insoluble when delivered between teeth and below the gum line during use. Additionally, saliva-soluble coatings preferably contain surfactants, mouth conditioners, chemotherapeutic ingredients and flavors that are released from the devices into the oral cavity, along with at least one sialagogue. For example, see U.S. Pat. Nos. 6,609,527 and 6,575,176.

As used herein, the term “crystal-free” refers to a smooth surface as distinguished from rough surfaces typical of crystalline coatings when observed through a 30× stereo zoom microscope. See U.S. Pat. No. 6,609,527. Generally, crystal-free coatings containing sialagogues indicate minimum flaking. Examples of suitable crystal-free, sialagogue-containing coating formulations for various interproximal dental devices of the invention are detailed in some of the Examples and Tables below.

As used herein, the term “inverse wax emulsion” defines a sialagogue-containing coating wherein the continuous phase is a suitable wax, such as microcrystalline wax, paraffin wax, beeswax, and the like, and the discontinuous phase is a surfactant. For example, emulsified as the discontinuous phase is a saliva-soluble surfactant solution of flavors, sweeteners, low-level active ingredients and other modifiers, along with sialagogues, which are released into the oral cavity in lesser amounts than the previously described “saliva-soluble, crystal-free coatings”. Upon disrupting by reason of the interproximal massaging action of the device, the continuous wax phase releases greater amounts of said ingredients than a simple wax mixture as commonly used in interproximal dental device manufacture. Formulations similar to the “saliva-soluble” coatings previously described can easily be incorporated into an inverse wax emulsion with sialagogues, as can simpler surfactant solutions as would be evident to one skilled in the art.

As used herein, the term “antimicrobial” includes various active ingredients that: control, disrupt and/or kill various microbiota associated with residual biofilms, which remain on tooth surfaces after flossing with the interproximal devices of the present invention. These include topical antimicrobials, such as: chlorhexidine digluconate (chlorhexidine), triclosan, benzylalkonium chloride, cetylpyridinium chloride, iodine, metronidazole and microbially active essential oils, such as thymol, menthol, etc.

A. Specifically, first generation antimicrobials include:
1. quaternary ammonium compounds such as benzethonium chloride, cetylpyridinium chloride,
2. phenolic compounds such as thymol and eucalyptol in a mixture of methyl salicylate, benzoic acid and boric acid and phenol,
3. natural extracts (flavor oils) known to possess antimicrobial properties, and
4. sanguinarine extract, alone or in combination with zinc chloride, or zinc chloride alone.

B. Second generation antimicrobials include: antibacterial agents with substantivity such as chlorhexidine, either free base or as the gluconate or other suitable salts, including alexidine, octenidine and stannous fluoride.

As used herein, the term “particulate abrasives” are defined as saliva soluble, semi-soluble and insoluble abrasive substances having a wide range of particle sizes and particle size distribution that are effective in physically removing, disrupting and controlling biofilms, when imbedded into the saliva soluble, coated, interproximal devices containing sialagogues of the present invention.

Other suitable particulate abrasives include various insoluble inorganics such as glass beads, and various insoluble organics such as particles of polyethylene, polypropylene, etc.

Particularly preferred inorganic particulate abrasives include various: (1) insoluble dental abrasives such as: pumice, silica, alumina, silicon dioxide, magnesium oxide, aluminum hydroxide, diatomaceous earth, sodium potassium aluminum silicate, zirconium silicate, calcium silicate, fumed silica, hydrated silica, and (2) soluble dental abrasives such as: dicalcium phosphate dihydrate, anhydrous dicalcium phosphate, sodium tripolyphosphate, calcium carbonate, etc. See also Tables below.

Particularly preferred “active” particulate abrasives include peroxides such as: carbamide peroxide, calcium peroxide, sodium perborate, sodium percarbonate, magnesium peroxide, sodium peroxide, etc.; phosphates such as: sodium hexametaphosphate, tricalcium phosphate, etc.; and pyrophosphates such as: tetrasodium pyrophosphate, tetrapotassium pyrophosphate, sodium acid pyrophosphate, calcium pyrophosphate, etc. See also Table 2 below.

See also the following relevant U.S. patents: U.S. Pat. Nos. 6,221,341; 3,491,776; 3,330,732; 3,699,979; 2,700,636; 5,220,932; 4,776,358; 5,718,251; 5,848,600; 5,787,758; and 5,765,576, which describe various oral care abrasives suitable for the present invention and which are incorporated herein by reference.

As used herein, the term “releasable particulate abrasive” is defined as the property whereby a particulate abrasive, which is imbedded into the saliva soluble base coating containing at least one sialagogue on the interproximal dental device, remains substantive to said base coating until the device is used, after which time the imbedded particulate abrasive in the base coating eventually separates from the device along with the base coating which eventually dissolves and releases the particulate abrasive into saliva. Thus, the particulate abrasive remains available interproximally and subgingivally to work with the interproximal device substrate, responding to biofilms encountered on interproximal, supragingival and subgingival tooth surfaces with physical-abrasive-type cleaning.

As used herein, the term “particulate abrasive load” is defined as the percent by weight of imbedded particulate abrasive contained on the coated interproximal dental device as a percent by weight of the device.

As used herein, the term “base coat interproximal device load” is defined as the percent by weight of the base coating containing at least one sialagogue contained on the interproximal device as a percent by weight of the coated interproximal device.

As used herein, the term “total coating load” is defined as the percent by weight of the saliva soluble base coating plus the particulate abrasive overcoating imbedded in said coating on the interproximal dental device as a percent by weight of the device.

As used herein, the term “biofilm-responsive” is defined as the property of dental devices to physically remove, disrupt and/or control biofilms and the microbiological burden associated with biofilms and to treat residual biofilms with sialagogues, while increasing and prolonging saliva flow and establishing anti-inflammatory activity interproximally and subgingivally.

As used herein, the term “fluidized bed” is defined as a means of converting solid particulate abrasives into an expanded, suspended, solvent-free mass that has many properties of a liquid. This mass of suspended particulate abrasive has zero angle of repose, seeks its own level, while assuming the shape of the containing vessel.

As used herein, the term “sequential fluidized beds” are defined as a means of converting solid particulate abrasives and solid particulate saliva soluble substances separately into expanded, suspended, solvent-free masses that have many properties of a liquid. These separate fluidized masses of suspended particulate abrasive and suspended solid, saliva soluble substances each have zero angle of repose and seek their own level, while assuming the shape of the containing vessel.

As used herein, the term “biofilm therapy interproximal devices” are defined as interproximal devices coated with sialagogues and saliva soluble coatings containing sialagogues that control, disrupt and physically remove biofilms, while increasing and prolonging saliva flow and delivering anti-inflammatory activity interproximally and subgingivally. In a preferred embodiment of the invention, the coated biofilm therapy interproximal devices of the invention include a SOFT ABRASIVES® overcoating that is also released, along with the sialagogues, during use to work with the substrate to help physically remove biofilms. Working these devices interproximally, massages interproximal soft tissues thereby increasing blood flow. The simultaneous release of sialagogues or various saliva soluble coatings containing sialagogues increases and prolongs saliva flow, and delivers anti-inflammatory activity interproximally.

As used herein, the term “micromesh dental floss” is defined as a monofilament dental tape, such as referenced in U.S. patent application Ser. No. 10/073,682, entitled, “Micromesh Interproximal Devices”, containing a sialagogue. Micromesh dental floss comprises a random: net, web or honeycomb-type integrated structure as distinguished from the more orderly monofilament and multifilament or woven structures used heretofore for interproximal devices. These micromesh structures are produced at low cost by integrating a rotating fibrillator device into a flat stretched film or tape producing operation, such as described in U.S. Pat. No. 5,578,373. A wide range of fibrillators are available to produce an almost endless array of micromesh structures.

As used herein, the term “flavorants” are defined as flavoring components suitable for emulsifying in saliva soluble coatings exemplified by the following substances: menthol, anise oil, benzaldehyde, bitter almond oil, camphor, cedar leaf oil, cinnamic aldehyde, cinnamon oil, citronella oil, clove oil, eucalyptol, heliotropine, lavender oil, mustard oil, peppermint oil, phenyl salicylate, pine oil, pine needle oil, rosemary oil, sassafras oil, spearmint oil, thyme oil, thymol, wintergreen oil, lemon and orange oils, vanillin, spice extracts and other flavoring oils generally regarded as safe (GRAS) by health authorities.

As used herein, the term “additional adjuvants” refers to additional ingredients that can be added to the emulsion/sialagogue mixture to provide color, or sweetening effects, as desired. Examples of suitable sweetening agents include sorbitol, sodium cyclamate, saccharine, commercial materials such as Nutrasweet® brand of aspartame and xylitol. Citric acid or acetic acid is often utilized as a flavor modifier and is generally used in amounts of about 1.0 to about 20 percent by weight, preferably about 2.0 percent to about 15 percent by weight.

As used herein, the term “buffering ingredient” refers to substances that may also be added to the flavored compositions of the invention in order to prevent natural degradation of the flavoring components or therapeutically active ingredients. Generally, the pH of these compositions is adjusted from about 3.5 to about 8, depending on the chemistry of the active ingredient most requiring protection. Buffering ingredients such as an alkali metal salt of a weak organic acid, for instance, sodium benzoate, sodium citrate, sodium phosphate, sodium bicarbonate or potassium tartrate is generally added in an amount of about 0.1 to about 1.0 percent by weight. Other buffering agents such as weak organic acids or salts of weak bases and strong acids such as boric acid, citric acid, ammonium chloride, etc., can also be used in similar concentrations.

As used herein, the term “stabilizers” refers to substances that are often added along with the flavorant to the coating compositions for additional control, such as:
(a) sodium benzoate, sodium or potassium sorbate, methyl paraben, propylparaben and others approved for ingestion, and
(b) chemical oxidative control substances, such as ethylene-diaminetetraacetic acid, BHA, BHT, propyl gallate and similar substances approved for ingestion. Concentration levels of these stabilizers comply with industry and regulatory standards.

As used herein, the term “shred resistant interproximal dental devices” describes the propensity of various monofilament interproximal devices to resist shredding, breaking or otherwise becoming discontinuous during flossing. Multifilament devices including texturized multifilament devices tend to be more prone to having individual filaments break and/or shred during flossing than monofilament tapes. On the other hand, certain monofilament tapes including PTFE tapes, and various extruded monofilament tapes, such as Fibaclean™ tape, tend to resist shredding and/or breaking during flossing due to their single monofilament construction combined with the low surface energy property of the tape. That the high density polyethylene (HDPE) monofilament devices, as described in referenced co-pending patent applications exhibit ultra shred resistant properties, ease of insertion and gentleness is surprising. The thin construction of these HDPE tapes and the break resistance attributed to their transverse orientation, combined with the “lubricants” in the saliva-soluble, crystal-free coatings of the devices, are believed to be primarily responsible for the exceptional: shred resistance, as well as for the surprising gentleness indicated.

As used herein, the term “SOFT ABRASIVES®” defines saliva-soluble and saliva-insoluble abrasive substances suitable for cooperating with the various interproximal substrates of the devices of the present invention to remove, control and disrupt biofilm, tartar and stained pellicle from tooth surfaces. SOFT ABRASIVES® include: tetrasodium pyrophosphate, calcium carbonate, dicalcium phosphate, silica, glass beads, polyethylene and polypropylene particles, pumice, titanium oxide, alumina, quartz, aluminum silicate, etc., at various particle sizes suitable for use in oral care. See U.S. Pat. No. 6,575,176.

As used herein, the term “whitening agents” for extrinsic stains refers to those substances which: (a) function as means of oxidation such as carbamide peroxide and hypochlorites, (b) function by interfering with calcium complex deposits such as tetrasodium phosphate or sodium hexametaphosphate (c) function as chelating agents, (d) function as abrasives such as the SOFT ABRASIVES® described above for stained pellicle disruption, control and removal.

As used herein, the term “cleaners” refers to essentially all surfactants suitable for use in the oral cavity and suitable for coating various interproximal dental substrates of the present invention.

As used herein, the phrase “chemotherapeutic ingredients” refers to those substances other than sialagogues suitable for addition to the coatings of the present invention that impart therapeutic effects to the oral cavity including antimicrobials; anti-tartar and anti-plaque substances; remineralizing, desensitizing, NSAID and antibiotic ingredients, and the like. Specific chemotherapeutic ingredients suitable for the present invention include: stannous fluoride, potassium nitrate, cetylpyridinium chloride (CPC), triclosan, metronidazole, chlorhexidine, aspirin and doxycycline.

As used herein, the phrase “substantially flake-free” refers to the propensity of the various interproximal dental device coatings of the present invention to resist flaking off during flexure. Flaking resistance is attributed to the crystal-free nature of the coatings and is based on the reduction by weight of the crystal-free coating after flexing, under suitably controlled and reproducible conditions, where an 18-inch piece of coated interproximal dental device is flexed for 30 seconds.

As used herein, the term “release value” refers to interproximal device coatings that are released from the substrate during use and is defined by measuring the level of coating remaining on 18-inches of the interproximal dental device after the device is used to thoroughly work between all teeth. The percent of the coating removed from the device during use establishes the release value.

As used herein, the term “formula modifiers” refers to those ingredients which are otherwise inactive as cleaners, abrasives or chemotherapeutic agents. Formula modifiers: (a) allow convenient control of the desired melt viscosity of the coating, (b) help provide the desired release rate in the mouth, (c) help provide for desired dispersability properties in the manufacturing process, and (d) improve mouthfeel for consumer acceptance.

As used herein, the term “viscosity control agents” includes those substances generally known in the food and consumer products, but not commonly used in devices, which are selected from natural and synthetic gums such as: carrageenan, gum tragacanth, methyl celluloses and derivatives there of such as hydroxymethyl methyl cellulose, polyvinyl pyrrolidone, and hydrophilic carboxyvinyl polymers such as those sold under the trademark Carbopol 934. Generally, about 0.01 percent to about 10 percent of one or more viscosity control agents is used. Often these substances are used as dry powders directly incorporated as a third phase into the melt-emulsion mixture. With appropriate control of the active water content, some or all of these dry viscosity agents could be substituted with pre-gelled viscosofiers containing no free water.

As used herein, the term “break strength” defines the force required in pounds to break a fiber or bundle of fibers or various monofilament tapes included with the interproximal dental devices of the present invention.

As used herein, the phrase “dimensional consistency” defines a physical property that, when measured along all three axes, is substantially the same, or when measured over an extended length of the substrate, remains substantially the same.

As used herein, the term “hand” defines the human tactile sensory response to a filament or group of filaments or monofilament dental tape. As in “soft hand” or feel of a fiber or fabric. Hand is a key consumer attribute and is required for holding and working certain monofilament interproximal dental devices of the invention.

As used herein, the term “tenacity” defines tensile strength expressed as force per unit density of an unstrained monofilament or multifilament interproximal device.

As used herein, the term “denier” defines the weight in grams of a multifilament bundle or a monofilament tape that is 9000 meters long.

As used herein, the term “low-denier” is defined as between about 250 and about 550.

As used herein, the term “fibrillation” is defined as a disrupting or penetrating of drawn films and tapes into a mesh like structure with numerous penetrations of the film or tape.

As used herein, the term “density” is defined as weight per unit volume.

As used herein, the term “elongation” is defined as the percent increase in length at break over starting length.

As used herein, “high density polyethylene interproximal device” is generally defined as a coated, low-denier, monofilament dental tape, indicating extraordinary gentleness and ease of insertion, that is slit and drawn from biaxially oriented, HDPE, blown film into substantially fibrillation-free, low denier dental tape referred to hereinafter as ParaFloss™. See U.S. copending patent application, Ser. No. 11/118,911.

In a preferred embodiment, ParaFloss™, the HDPE resin modified with certain antifibrillation agents, is blown extruded making a biaxially oriented, high density polyethylene (HDPE) film having a density of at least about 0.940, a melt index from between about 0.5 and about 10, wherein the HDPE is drawn longitudinally to a degree from between about 5:1 and about 15:1, thereby producing a biaxially oriented; blown HDPE film, preferably having a machine to transverse ratio of from about 1.6:1 to about 2.0:1.

This blown HDPE film having a thickness between about 3 and about 6 mils is then slit and drawn into dental tape having a thickness from between about 0.6 and about 3.0 and a width from between about 20 and about 80 mils. The film is slit and drawn at speeds ranging from between about 200 ft/minute and about 500 ft/minute. This tape substantially resists fibrillation during coating, bobbin winding and flossing.

### DETAILED DESCRIPTION OF THE INVENTION

There are five oral care, biofilm-responsive factors delivered by the sialagogue-containing dental devices of the invention interproximally and subgingivally. These include abrasion, removal of precursors to plaque, altering the attachment of plaque to a surface, enhancing saliva flow, and anti-inflammatory activity:
1. Abrasive removal of the plaque film, once it has firmly adhered to the tooth surface, is the only totally effective cleansing mechanism. Again, professional dental hygiene is most effective, but recently, a number of special abrasive toothpastes have been accepted by dental organizations for partially removing supragingival adhered plaque and the tartar which subsequently forms from the plaque. Heretofore, interproximal plaque could only be removed by mechanical means such as flossing and/or by use of appropriately shaped dental stimulators;
2. Removal of plaque precursors requires the reduction of food sources and building blocks required for the bacteria to synthesize the mucopolysaccharides which polymerize into the plaque film. Going far back into the chain of events leading to plaque formation and interrupting the chain has much to commend it as a sound oral hygiene strategy. However, for this technique to be effective, the plaque building blocks must be interrupted periodically throughout the mouth, especially at the site of plaque buildup and if possible just below the tooth-gum interface and interproximally. Such disruption is disclosed and claimed in U.S. Pat. No. 4,958,479, the disclosure of which is hereby incorporated herein by reference. Most other oral hygiene preparations described above fall short on “frequency-of-use” basis, abrasion and cleaning. For reference see L. Menaker, “The Biological Basis of Dental Caries,” Chapters 5, 11, 12, 14, 16 and 18, Harper and Row (1980);
3. As to altering attachment of plaque, it has now been found that the cleaning and coating compositions described below can be incorporated into dental floss of specified construction at surprisingly high concentrations; considering that the compositions of the present invention are not soluble in the floss. Secondly, floss so treated will “release” these compositions during flossing and chemically cleanse the area of plaque and plaque precursors, bacteria, etc., while coating teeth and gum surfaces with a plaque matrix disrupting substance. The release of these substances is particularly effective in disrupting, for prolonged periods, the plaque matrix on these interproximal sites. The cleaning that results from the composition released from the floss also takes place on those interproximal surfaces brushing does not reach. This chemical cleansing and matrix disruption adds a new dimension to flossing beyond the physical removal of debris from these surfaces;
4. Enhancing saliva flow by increasing and prolonging saliva flow through the release of sialagogues; and
5. Anti-inflammatory activity including inhibiting pro-inflammatory cytokines and free radicals and targeting prostaglandins, which are involved in late phase of acute inflammation and pain perception.
   In addition, there are three unexpected sensory-responsive factors delivered by the devices of the present invention interproximally and subgingivally, as detailed below:
1. Increase of instantaneous, during-use pleasure which increases the interproximal devices use frequency, duration of use and the encouragement of effective use techniques. This includes the introduction of pleasant flavors, mouthfeel and salivation which typically accompanies the consumption of pleasant foods, treats and confections. Dental devices coated with sialagogues alone or combined with surfactants, emulsions or MICRODENT®, stimulate the non-olfactory (smell or aroma), non-flavor (i.e., bitter, sweet, sour and salty) sensory receptors. This produces a surprising increase of during-use pleasure of otherwise unpleasant interproximal devices far beyond that typically expected from typical flavors or cooling ingredients such as menthol.
2. Increase of post-use pleasure to re-enforce the desire to floss or otherwise clean the interproximal spaces regularly. Dental devices coated with sialagogues are surprising in their longevity of effect, especially when delivered gingivally. The reminder that “I did something good for myself” over 10 to 15 minutes after application is a surprisingly strong reinforcement of the psychological conditioning that produces good oral care habits. This is especially true since the sensory-response is from the interproximal spaces where the sialagogue was delivered.
3. Reduction of pain without the excessive numbing of traditional mouth-pain ingredients like benzocaine and lidocaine. Benzocaine and lidocaine create an unpleasant numbing perception while sialagogues have a simultaneous tingling sensation that actually makes the affected areas feel more “alive” even while reducing the perception of normal pain generation associated with use of interproximal devices. This surprising effect is particularly observed when the sialagogues are introduced directly to the gingival surfaces at the same time the pressure induced pain is generated.

Biofilm-responsive interproximal dental devices containing a sialagogue or a saliva soluble coating and at least one sialagogue include: multifilament dental flosses, texturized multifilament dental devices, monofilament dental devices, dental flossers, dental picks, dental massagers, proxy brushes and interdental irrigators.

Regular use of various devices of the present invention provides a unique combination of: mechanical action, detersive action, surface modification and chemotherapy with increased and prolonged saliva flow attributed to the sialagogues, resulting in:
(a) disruption of subgingival microflora, and
(b) removal of interproximal supragingival plaque (biofilm).

Subgingival chemotherapeutic disruption of microflora is achieved by the unique combination of:
(a) chemical cleansing with surfactants released from the floss of the present invention,
(b) prolonged modification of the surface chemistry of the microflora by the coating materials released, e.g., silicone emulsions, released from the floss, and
(c) alteration of microflora with various active ingredients contained in the load and released during flossing.

Subgingival mechanical disruption of microflora is achieved by the unique combination of:
(a) physical disruption by the device substrate,
(b) abrasive, disruption with abrasives released from the substrate including: silica, dicalcium phosphate, pyrophosphates, etc., at concentrations up to 40 mg/yd; and
(c) surfactant disruption resulting from the release of surfactants during use.

Chemotherapeutic removal of supragingival plaque (biofilm) is achieved by the unique combination of:
(a) chemical cleansing with surfactants released from the device,
(b) modification of the surface chemistry of the plaque with coating materials, e.g., silicone emulsions, and
(c) alteration of the plaque with various active ingredients contained in the load and released during use including: tetrasodium pyrophosphate, tetrapotassium pyrophosphate, etc.

Mechanical removal of supragingival plaque is achieved by the unique combination of:
(a) physical removal by the device substrate,
(b) abrasive removal by the abrasives released from the device including: silica, dicalcium phosphate, pyrophosphates, etc., and
(c) cleansing resulting from the release of surfactants during flossing.

The physical improvements in various devices such as monofilament tapes of the present invention include:
A “reduced-cutting-edge” effect which is achieved by:
1. Extruding:
   (a) rounded edged, or
   (b) multiple rounded surfaces at and edge, or
   (c) an edge so thin and flexible that it bends or folds on contract with soft tissue; or
2. Slitting extrusion-blown films while they are undergoing various drawing processes.

These various extruded or slit/drawn edges cushion the impact of these modified monofilament tapes on soft tissue. The cushioning tape edges are achieved during extrusion of the monofilament tape prior to drawing, by using various shaped extrusion dies or by slitting extrusion-blown films during drawing. These customized dies deliver various changes to the monofilament tape cutting edge dimensions, while the slitting/drawing process results in “rounded” edges.

As referenced above, the modified monofilament tapes of the present invention include physical improvements made to coated monofilament dental tape, particularly wherein said improvement is selected from the group consisting of:
(a) a reduced cutting edge, wherein during monofilament tape extrusion, or films extrusion/drawing/slitting, at least one lateral edge of said tape is formed into various shapes selected from the group consisting of: rounded shapes, multiple rounded shapes, thin flexible edges, multiple edges and combinations thereof,
(b) feathered lateral tape edges comprising multiple fingers of varying densities and varying lengths introduced by various continuous cutting means applied to at least one edge of said tape after extrusion and prior to coating,
(c) self-indicating features incorporated into said coatings using a combination of colored tapes and colored tape coatings wherein various scribes applied to the coatings remove portions of the coating to form a visual predetermined design, and
(d) combinations thereof.

In a preferred embodiment, the present invention is directed to biofilm-responsive, saliva soluble, coated dental devices containing an emulsion and at least one sialagogue overcoated with soft abrasives which are released during flossing and are suitable for physical-abrasive-type removal and disruption of biofilms and chemotherapeutic treatment of biofilms that form on interproximal, supragingival and subgingival tooth surfaces not reachable by brushing or rinsing.

The saliva soluble, coated interproximal dental devices of the present invention containing at least one sialagogue are preferably overcoated with an imbedded particulate abrasive that remains imbedded in the saliva soluble, base coating until said base coating is eventually released from the interproximal dental device during use. See FIGS. 1 through 3 of the drawings.

For example, during use, at the outset, the imbedded particulate abrasive overcoating functions as a “soft” abrasive version of an oral-type sandpaper removing and disrupting biofilms and stained pellicle. Essentially the first pass through an interproximal space by the imbedded particulate overcoated, interproximal dental device results in a gentle “sandpaper” abrasive effect on the biofilms present, which effect is eventually followed by dissolving and/or breaking up of the saliva soluble, base coating and release of the imbedded particulate overcoating, wherein the base coating contains an emulsion, at least one sialagogue and particulate abrasive.

After the saliva soluble base coating is released, the soft abrasive particulate overcoating works in conjunction with the interproximal device substrate to continue to remove and disrupt biofilms until the particulate abrasive is flushed away and/or dissolved by saliva. That is, the released particulate abrasive cooperates with the interproximal substrate as the device is being worked over interproximal, supragingival and subgingival surfaces, while continuing to deliver emulsion coatings, at least one sialagogue and flavorant to residual biofilms on interproximal, supragingival and subgingival tooth surfaces.

The physical-abrasive-type cleaning and disruption of biofilms achieved with the various imbedded particulate soft abrasives overcoated interproximal dental devices of the present invention containing at least one sialagogue continues:
(a) until the spent device is removed from the interproximal site and working of the area is discontinued,
(b) as the saliva soluble emulsion coats the interproximal surfaces,
(c) as the sialagogue(s) contained in the emulsion coating increase and prolong saliva flow and/or deliver anti-inflammatory activity,
(d) until the particulate abrasive dissolves and/or is washed away by saliva, and/or
(e) until the biofilm is physically removed or disrupted.

The physical-abrasive-type cleaning and disruption of biofilms with the imbedded particulate abrasive overcoated devices of the present invention are simultaneously supplemented with: (1) a biofilm-responsive coating contained in the saliva soluble base coating, (2) at least one sialagogue, and (3) a flavorant used to introduce flavor, mouth feel, conditioning, etc., attributes to interproximal sites. In these versions, these substances are released onto interproximal tooth surfaces and soft tissue during use along with the saliva soluble base coating to help disrupt and control the microflora associated with residual biofilm not removed during use of the device and to simultaneously increase and prolong saliva flow and/or to simultaneously deliver anti-inflammatory activity to interproximal sites. See FIGS. 1 through 3.

Surprisingly, the particulate abrasive overcoating imbedded in the saliva soluble base coating on the dental devices of the present invention exhibits unexpected gentleness along with lower than expected abrasivity which, for purposes of the present invention, allows more abrasive particulates to be used in the overcoating, such as pumice, alumina, silica, etc. This “soft abrasive” effect is attributed in part to the cushion effect contributed by the saliva soluble base coating, provided the imbedded particulate abrasive. That is, the base coating containing the partially imbedded particulate abrasive tends to cushion the impact of the exposed portion of the abrasive particulate onto tooth surfaces and soft tissue during use. See FIGS. 1 through 3. When the abrasive/saliva soluble coating mixture is released from the device substrate during use, the base coating tends to help lubricate the particulate abrasive/micromesh combination further reducing the abrasivity of the particulate soft abrasive on tooth surfaces and soft tissue, while simultaneously increasing and prolonging saliva flow, and imparting anti-inflammatory activity to interproximal sites.

### BRIEF DESCRIPTION OF THE DRAWINGS

FIGS. 1 through 3 are schematic illustrations of dental floss and dental tape interproximal devices of the present invention being used to remove, disrupt and control biofilms physically and to topically treat residual biofilm remaining after flossing with a sialagogue or a substantive emulsion coating containing a sialagogue.

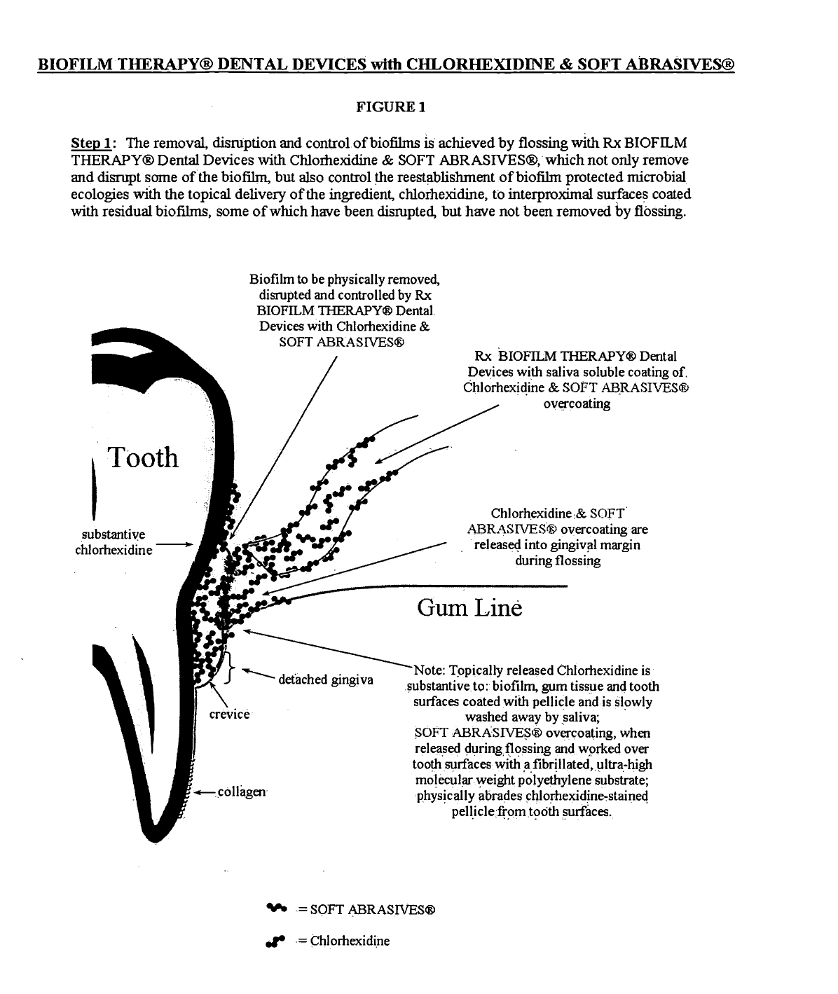

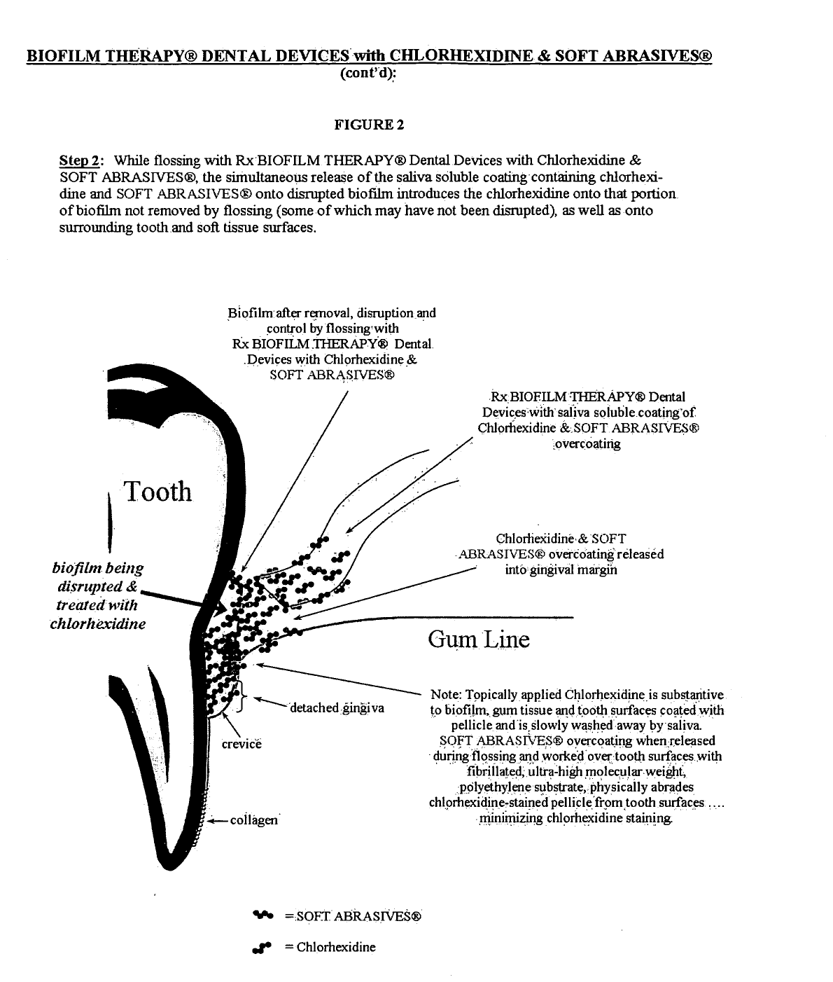

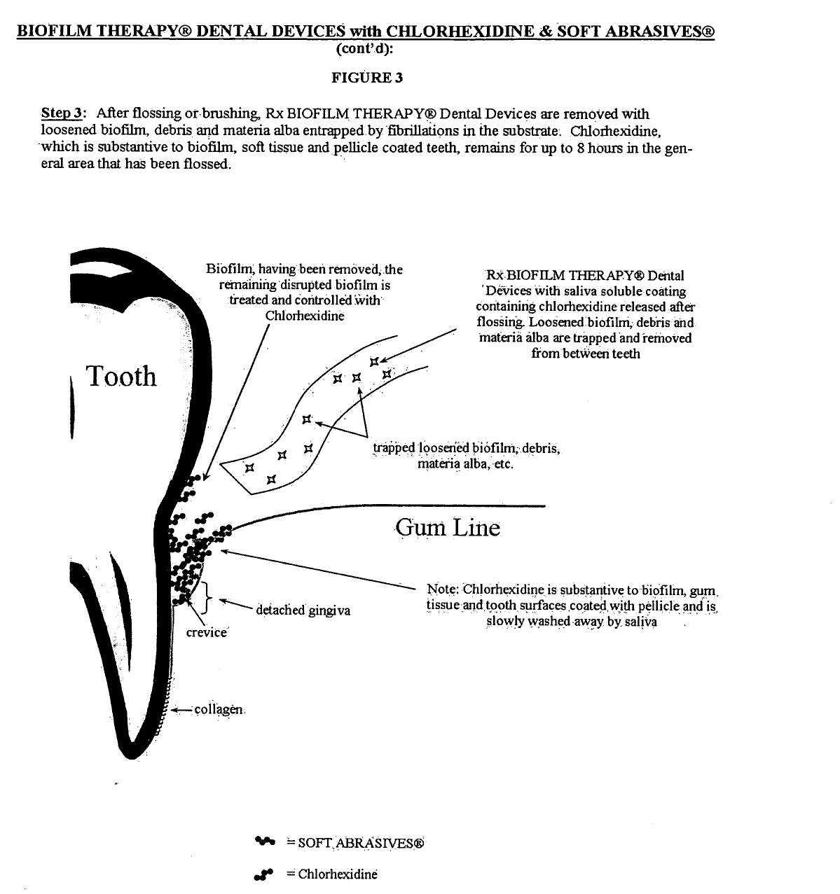

Referring to FIGS. 1 through 3, the following mechanism of action is illustrated:

A. During Use:

Step 1: The saliva soluble base coat at 75 mg/yd containing 32 mg/yd emulsion with flavor and a sialagogue is released from the fibrillated tape, along with the overcoating of 8 mg/yd SOFT ABRASIVES®.
Step 2: The released saliva soluble coating proceeds to clean and coat teeth and soft tissue surfaces with flavor and sialagogue before dissipating into the saliva flow.
Step 3: The insoluble SOFT ABRASIVES® particulate overcoating, which has released from the substrate, prior to being flushed away by the saliva, is worked over interproximal tooth surfaces, subgingivally and supragingivally, by the substrate that is now substantially free of the saliva soluble coating.
   This SOFT ABRASIVES® containing substrate combination removes, disrupts and controls biofilm until such time as the SOFT ABRASIVES® are flushed away by the saliva and the tape is removed.
Step 4: The substantive emulsion containing a sialagogue attaches to residual biofilm not removed by flossing, as well as to the pellicle present on tooth surfaces and increases and prolongs saliva flow and establishes anti-inflammatory activity interproximally and subgingivally.
   B. After Use:
Step 1: The spent substrate, which is removed from interproximal spaces, contains entrapped:
   loosened biofilm,
   loosened stained pellicle,
   stained microbiota
   food particles,
   debris,
   materia alba, etc.
Step 2: The substantive emulsion coating containing flavor and sialagogue remains substantive, disrupting and controlling the residual biofilm and increasing saliva flow for several hours.
Step 3: The mouth “feels and tastes” fresh and clean with enhanced saliva flow.

### Coatings and Overcoatings of the Invention

The various interproximal devices of the present invention can contain sialagogues alone or a broad range of saliva soluble coating substances containing a surfactant/sialagogue mixture or an emulsion/sialagogue mixture. These sialagogues are best loaded onto and/or into the interproximal device structure by one of four loading means. Specifically:
1. The high melt viscosity mixtures and emulsions containing the emulsion/sialagogue mixture are loaded onto and/or into various interproximal devices by compression means;
2. The medium melt viscosity mixtures and emulsions containing the emulsion/sialagogue mixture are loaded onto and/or into various interproximal devices by injection loading means; and
3. The low melt viscosity mixtures and emulsions containing the emulsion/sialagogue mixture are loaded onto and/or into various interproximal devices by contact loading means.
4. Various versions of 1-3 above and sialagogues, per se, can be applied to the various dental devices by dipping the various interproximal devices into the coatings. These include proxy brushes, flossers and dental picks.

When coating certain interproximal devices with only a sialagogue in a carrier such as ethanol; dipping, soaking and/or spraying with a liquid carrier/sialagogue mixture is preferred.

The improved interproximal devices of the present invention contain base coatings with emulsion/sialagogue mixtures that: (a) comprise from 10 to 120% by weight of various interproximal device substrates, (b) are preferably saliva soluble and (c) in a preferred embodiment are crystal free, and accordingly, exhibit a minimum of flaking. Some of these base coatings are released in total into the oral cavity during use.

In a preferred embodiment, these base coatings contain in addition to the emulsion and a sialagogue, other ingredients such as: (a) antimicrobials, (b) SOFT ABRASIVES® that work with the substrate to help physically remove biofilm (plaque) from interproximal, supragingival and subgingival surfaces, (c) other chemotherapeutic ingredients affecting oral health, (d) cleaners that introduce detersive effects into the areas worked by the various devices, and (e) mouth conditioners and flavorants. These base coatings are particularly adapted to loading into and/or onto the dental flosses and dental tapes using the compression, injection or contact loading means described above to produce the innovative interproximal devices of the present invention. Other interproximal devices such as flossers, proxy brushes, dental picks and dental massagers are adapted to being coated with these base coatings by various contact loading means and dipping loading means.

The particulate abrasives and other saliva soluble particulate substances of the present invention are overcoated onto the coated interproximal dental device base coatings as solid materials substantially free from solvents.

A preferred method of imbedding particulate abrasive overcoatings and saliva soluble particulate overcoatings into the base coat of various flossing devices is by means of a series of innovative fluidized bed systems such as described in several of the co-pending patent applications.

The overcoatings of particulate abrasive and various saliva soluble particulate substances containing flavorants and/or mouth conditioners and/or chemotherapeutic substances can include a broad range of these substances. For example, ratios of particulate abrasives to saliva soluble substances such as nonionic surfactants (PLURONIC®), emulsions such as MICRODENT® and/or ULTRAMULSION® and/or polyols such as PEG in these hi-impact particulate overcoatings can range from 10:90 to 90:10.

The innovative fluidized bed coating process referenced above is most effective in imbedding:
(1) particulate abrasive loads between about 2 and about 45 percent by weight into the saliva soluble, coated device, and
(2) particulate, saliva soluble loads between about 2 and about 45% by weight into the coated device.

It has been discovered that in order to produce a sialagogue containing coated and overcoated interproximal dental device effective in controlling, disrupting and removing biofilms, it is necessary: (1) to imbed particulate abrasive loads at between about 10 and 34 percent by weight of the device, (2) to restrict the average particle size of the imbedded particulate abrasive to between about 7 microns and about 200 microns, (3) to restrict the particle size distributions of the imbedded particulate abrasive to from between about 5 microns and about 300 microns, and (4) to imbed the particulate abrasive into the saliva soluble liquid base coating under a high velocity charge from several nozzle means positioned at 90° to the traverse of the coated interproximal device through the particulate coating chamber, thereby maximizing the impingement of the particulate abrasive into the base coating.

Overcoating sialagogue containing coated interproximal devices of the invention with saliva soluble particulate can be carried out by imparting a static charge to the saliva soluble particulate prior to discharge from the nozzle means. Means are provided for grounding the liquid, base, coated interproximal device in order to receive the charged saliva soluble particulate. Alternatively, saliva soluble particulate can be imbedded into liquid base coatings on various interproximal devices of the invention by various spraying means.

In addition to various types of fluidized bed/nozzle arrangements, the particulate abrasive overcoatings can be imbedded into the coated interproximal devices by several other means for impinging particulate abrasives onto liquid coated devices. These include various powder coating processes including fluidized bed, plastic frame-spraying, electrostatic spraying and sonic spraying. In the latter, sound waves are used to suspend the particulate abrasives before introducing the fluidized particulate abrasive into a nozzle means.

Other particulate abrasive overcoating processes are described in U.S. Pat. Nos. 6,037,019; 3,848,363; 3,892,908; 4,024,295; 4,612,242; 5,163,975; 5,232,775; 5,273,782; 55,389,434; 5,658,510; 2,640,002; 3,093,501; 2,689,808; 2,640,001 and 5,194,297. These can be adapted to particulate abrasive impingement on coated interproximal devices as taught by the present invention and are incorporated herein by reference.

Particularly preferred particulate overcoating means include various Nordson® automatic powder coating systems such as the Nordson® Tribomatic II powder coating system, which includes various Nordson® powder pumps, as well as ITW Gema Powder coating systems including their Easysystem™ and Electrostatic Equipment Co's 7R FLEXICOAT® system.

The particulate overcoating of the invention can be affected with various other means for delivering particulate to the saliva soluble liquid base coating. For example, the particulate can be introduced by a simple screening technique where the particulate drops from the screening means onto the liquid means onto the liquid base-coated interproximal dental device.

The preferred means of the invention for overcoating includes a fluidized bed in combination with a nozzle means. This combination provides the most uniform overcoatings while controlling the extent of the particulate imbedding into the liquid base coating.

Crystal-free, substantially flake-free, base coatings containing an emulsion, and a sialagogue suitable for various interproximal flossing dental devices are described in Illustrative Examples 1 through 18 in Table 1 below.

In a preferred embodiment, the improved interproximal dental devices of the present invention contain a wide range of saliva soluble coatings containing sialagogues including saliva soluble waxes, contact and compression coatings that: (a) comprise from 10 to 120% by weight of the substrate, (b) are saliva soluble, and (c) in a preferred embodiment are crystal free, and accordingly, exhibit a minimum of flaking. Preferably, these saliva soluble coatings containing sialagogues are released in total into the oral cavity during flossing.

In a preferred embodiment, these various sialagogue-containing, saliva soluble coatings contain ingredients such as: (a) SOFT ABRASIVES® that work with the substrate to help physically control, disrupt and remove biofilms (plaque) from interproximal and subgingival surfaces, (b) chemotherapeutic ingredients for controlling biofilms, (c) cleaners that introduce detersive effects into the areas flossed, and (d) mouth conditioners. These coatings are particularly adapted for coating onto various interproximal devices using wax coating, contact coating, compression coating, and various dipping/spraying means such as described herein to produce easy-to-use, interproximal dental devices of the present invention.

In addition to devices coated with sialagogues, per sé, it has been discovered that the substantivity of certain emulsion coatings containing sialagogues, compression and/or contact coating onto interproximal dental devices of the present invention can be enhanced such that during flexure of the coated devices, these enhanced coatings remain substantive to said devices and resist cracking, fracturing and flaking off. Specifically, it has been observed that most compression and contact coated, flexible surfaces, especially those formulated to be saliva soluble and to carry effective quantities of abrasives, cleaners, surfactants, and chemotherapeutic agents; fracture along crystal faces during flexure of the device and tend to prematurely release the ingredients from the flexible device surface by cracking, chipping, flaking and/or falling off, etc. In response to these observations, it has been unexpectedly found that the addition of certain substances to various emulsion compression and contact coatings at relatively modest levels reduces crystal formation while simultaneously enhancing the coating's substantivity to these various interproximal dental devices when subjected to flexure. These properties thereby impart outstanding flake resistance and release values to said compression and contact coated interproximal dental devices of the invention.

Waxed coatings containing sialagogues are applied to various interproximal devices of the invention utilizing standard waxing means presently used for multifilament and monofilament substrates. See U.S. Pat. Nos. 4,911,927; 5,165,913; 5,098,711; 5,711,935; 6,545,077; 2,667,443; 5,967,153; 5,908,039 and 5,830,495, which are incorporated herein by reference.

Those coating additives that reduce, control and/or eliminate crystal formation and enhance the substantivity of the loaded sialagogue-containing coating to flexible interproximal devices of the invention when added to these coatings at modest levels include certain aliphatic, long chain, fatty alcohols having from between about 10 and 30 carbon atoms and/or various liquid surfactants such as polyethylene glycol sorbitan dialiphatic esters.

Suitable aliphatic, long chain, fatty alcohols for the crystal-free coatings of the present invention can be represented by the structural formula ROH, wherein R represents a long chain alkyl group having from 20 to 30 carbon atoms. Specific examples include:

| | | |
|---|---|---|
| 1-decanol | 1-heptadecanol | 1-pentacosanol |
| 1 undecanol | 1-octadecanol | 1-hexacosanol |
| 1-dodecanol | 1-nonadecanol | 1-heptacosanol |
| 1-tetradecanol | 1-decosanol | 1-octacosanol |
| 1-pentadecanol | 1-henticosanol | 1-nonacosanol |
| 1-hexadecanol | 1-tricosanol | 1-triacosanol |
| 1-tetracosanol, | | |
| and mixtures thereof. | | |

Naturally occurring mixtures with substantial quantities of these fatty alcohols, or isomers thereof; including those chemically derived from natural sources also constitute suitable sources of aliphatic, long chain fatty alcohols for the purpose of this invention.

The long chain fatty alcohols can be purchased commercially from Stepan, Procter & Gamble and Aldrich Chemical Co. and a variety of companies processing vegetable and animal derived fatty alcohols.

Suitable liquid surfactants for the saliva-soluble, crystal-free coatings of the present invention include polyoxyethylene glycol sorbitan mono- and di-aliphatic esters represented by the general formula: 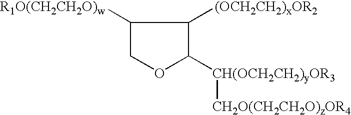
wherein R₁, R₂, R₃, R₄ and H or aliphatic acyl groups having from between about 10 and 30 carbon atoms, and the sum of w, x, y, and z is from between about 20 and about 80.
These liquid surfactants are available under the trade name Emsorb®, Span®, Tween® from Cognis, N.A. and ICI. Specific examples of these include:
PEG 20 sorbitan monooleate (Tween® 80, ICI); PEG 40 sorbitan monostearate (SPAN 60 ICI) and PEG 40 sorbitan diisostearate (Eumulgin® SDI 40, Cognis N.A.).
Suitable solid surfactants that contribute to a solid-at-room-temperature coating matrix include:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and polyoxyethylene compound having as a hydrophobe a polyoxypropylene polymer of at least 1200 molecular weight (these surfactants are generally described as poloxamers; specific examples are described in the Examples below) as poloxamer 407 and poloxamer 388,
soap powder, and
mixtures thereof.

These same solid surfactants can, when desired, aid the proper emulsification of any of the waxes and other discontinuous, hydrophobic constituents in the formula.

Certain wax, contact and compression coated interproximal dental devices of the present invention feature ultra shred-resistance combined with superior gentleness. The coatings released during use can deliver cleaners, sialagogues, chemotherapeutic ingredients, etc., along with SOFT ABRASIVES® between teeth and below the gum line. These substances also collectively impart lubricity to the interproximal devices during flossing. This coated substrate combines with the SOFT ABRASIVES® released during use to gently control, disrupt and remove biofilm, tartar and stained pellicle from tooth surfaces between teeth and below the gum line.

The wax, contact and compression coated interproximal substrates, in combination with sialagogues and SOFT ABRASIVES® loaded on the coated interproximal device which is later released during flossing, creates a perceptible impression that the device is working to control, disrupt and remove biofilm, tartar, stained pellicle, debris, etc., as the device is being worked between teeth. This “it's working” perception in combination with sialagogue impacted saliva flow is a critical “compliance” advantage over most interproximal dental devices available commercially.

The preferred saliva-soluble, substantially crystal-free emulsion coatings of the invention can contain various cleaners, SOFT ABRASIVES®, chemotherapeutic ingredients, as well as at least one sialagogue, flavorants, etc. These latter substances tend to leave a lasting (substantive) coating on surfaces in the oral cavity that imparts increased saliva flow for prolonged periods, along with a refreshing, just-brushed feeling that encourages and motivates regular flossing, particularly after meals and snacks while away-from-home. Particularly preferred mouth conditioners include sialagogues in various MICRODENT® and ULTRAMULSION® emulsions such as described in U.S. Pat. Nos. 4,911,927; 4,950,479; 5,032,387; 5,098,711; 5,165,913; 5,538,667; 5,645,841; 5,561,959 and 5,665,374.

The mechanical action of the coated interproximal dental substrate, in combination with sialagogues and SOFT ABRASIVES® released from the various coatings during use is further supplemented by the various cleaners including surfactants also released with these coatings during use. These released cleaners are readily soluble in the saliva and interproximal fluids and produce a detersive effect in the interproximal and subgingival regions. This detersive effect is critical in helping to loosen biofilm, tartar, stain residue and other debris from tooth surfaces.

In addition to the sialagogues, cleaners and SOFT ABRASIVES® described above, the wax, contact and compression coatings added to the interproximal dental substrates can also contain various chemotherapeutic ingredients including anti-biofilm and anti-tartar agents, as well as active ingredients such as antimicrobials, biofilm attachment altering ingredients such as MICRODENT® and ULTRAMULSION®, and anti-tartar ingredients such as the pyrophosphates. All of these can be delivered interproximally and subgingivally by the coated interproximal dental devices of the present invention during use.

In a preferred embodiment of the invention, the saliva soluble, coated, innovative, biaxially oriented, HDPE interproximal devices (ParaFloss™) of the present invention containing sialagogues are designed to replace:
a. commercial monofilament dental tapes such as P&G's Glide® and Comfort Plus®, J&J's easySLIDE®, Colgate's Total®, Oral-B's Satin® Floss, Jordan's Fibaclean®, as well as
b. commercial waxed, multifilament dental flosses such as J&J's REACH® Waxed Flosses, including the multiplicity of Private Label equivalents, and Ranir's Hi-Tech® Floss.

The broad appeal of coated HDPE interproximal devices (ParaFloss™) wax, contact and/or compression coated with various coatings containing sialagogues to certain monofilament tape users and to certain multifilament floss users represents a major advance in commercial interproximal dental devices that promises to:
a. improve overall interproximal cleaning,
b. improve the disruption, control and/or removal of biofilm, tartar, stained pellicle, debris, etc., from tooth surfaces,
c. increase saliva flow and prolong the duration of increased saliva flow,
d. deliver anti-inflammatory activity interproximally and subgingivally, and
e. encourage flossing compliance.

Accordingly, one embodiment of the present invention is directed to shred-resistant, oriented, low-denier, blown, slit and drawn HDPE interproximal devices, which are wax, contact or compression coated with liquid coatings including waxes, mixtures and/or emulsions containing: sialagogues, cleaners, chemotherapeutic ingredients and, in some instances, SOFT ABRASIVES® at from between about 10 and about 120 mg/yd.

Advantageously, when the various coatings contain insoluble SOFT ABRASIVES® of an appropriate particle size, these abrasives, once released, tend to compliment the HDPE structure during flossing to gently scrub biofilm, tartar and stained pellicle off tooth surfaces.

The various coated HDPE dental devices of the present invention are a most effective means for controlling disrupting and removing biofilms while increasing and prolonging saliva flow and for delivering chemotherapeutic substances to interproximal and subgingival areas of the oral cavity. The sialagogues contained in the coatings loaded onto the interproximal dental devices of the present invention can be delivered to specific interproximal and subgingival sites during using.

Various chemotherapeutic agents suitable for inclusion in the coatings of the dental devices of the present invention include:
1. anti-plaque and/or anti-tartar substances such as MICRODENT®, ULTRAMULSION®, tetrasodium pyrophosphate (TSPP);
2. tetrapotassium pyrophosphate, sodium hexametaphosphate, etc.;
3. first generation anti-biofilm agents which are antibacterial agents with limited substantivity such as oxygenating compounds, quaternary ammonium compounds, phenolic compounds and plant alkaloids, including:
   a. quaternary ammonium compounds such as benzethonium chloride and cetylpyridinium chloride (CPC),
   b. phenolic compounds such as thymol and phenol, methyl salicylate and other compositions such as benzoic acid and boric acid,
   c. natural extracts (flavor oils) known to possess antimicrobial properties including eucalyptol,
   d. sanguinaria extract, alone or in combination with zinc chloride, or zinc chloride alone,
   e. triclosan, and
   f. iodine;
4. second generation agents which are antibacterial agents with substantivity such as chlorhexidine, either free base or as the gluconate or other suitable salt, alexidine, octenidine and stannous fluoride. The treatment of the oral cavity with stannous fluoride or chlorhexidine antimicrobial containing HDPE dental devices are preferred embodiments of the present invention;
5. desensitizing agents, antibiotics, anti-thrush agents, substances to neutralize candida sp. yeasts, anti-caries agents, antimicrobials, COX-2 agents, etc.;
6. dry mouth relieving agents;
7. NSAIDs including aspirin;
8. antibiotics including doxycycline, tetracycline and minocycline; and
9. metronidazole.
   Certain petroleum waxes are suitable and preferred additional additives for the crystal-free coating of the present invention. These include any of a range of relatively high molecular weight hydrocarbons (approximately C₁₆ to C₅₀), solid at room temperature, derived from the higher-boiling petroleum fractions. There are three basic categories of petroleum wax: paraffin (crystalline), microcrystalline, and petroleum. Paraffin waxes are produced from the lighter lube oil distillates, generally by chilling the oil and filtering the crystallized wax they have a melting point range between 48° C. (118° F.) and 71° C. (160° F). Fully refined paraffin waxes are dry, hard, and capable of imparting good gloss. Microcrystalline waxes are produced from heavier lube distillates arid residue (one bottoms) usually by a combination of solvent dilution and chilling. They differ from paraffin waxes in having poorly defined crystalline structure, darker color, higher viscosity, and higher melting points—ranging from 63° C. (148° F.) to 93° C. (200° F). The microcrystalline grades also vary much more widely than paraffins in their physical characteristics: some are ductile and others are brittle or crumble easily.

Petrolatum is derived from heavy residual lube stock by propane dilution and filtering or centrifuging. It is microcrystalline in character and semi-solid at room temperature. There are also heavier grades for industrial applications, such as corrosion preventives, carbon paper, and butcher's wrap. Traditionally, the terms slack wax, scale wax and refined wax were used to indicate limitations on oil content. Today, these classifications are less exact in their meanings, especially in the distinction between slack wax and scale wax. Natural waxes such as beeswax and carnauba wax are also suitable and may provide specifically desired properties.

Examples of saliva-insoluble formula modifiers suitable for the various coatings, include:

microcrystalline waxes,
paraffin wax,
carnauba, beeswax and other natural waxes,
animal and vegetable fats and oils, and
low-melt point, orally suitable polymers and copolymers.

Examples of saliva-soluble formula modifiers include so-called water soluble waxes, such as:
liquid polyethylene glycols,
solid polyethylene glycols,
liquid polyethylene glycols,
solid polypropylene glycols, and
triacetin.

Examples of low-melt temperature, water-soluble polymers, include:
hydroxyethylcellulose,
hydroxypropylcellulose,
carboxy derivatives of cellulose, and
orally suitable saliva gelling or water-soluble copolymers of various resins.

Suitable emulsions of various coating substances in various surfactant continuous phases include those described and claimed in the various MICRODENT® and ULTRAMULSION® U.S. patents including U.S. Pat. Nos. 4,911,927; 4,950,479; 5,032,387; 5,098,711; 5,165,713; 5,538,667; 5,645,841; 5,561,959 and 5,665,374, all of which are hereby incorporated by reference.

Examples of suitable surfactants include:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and
polyoxyethylene compound having as a hydrophobe a polyoxypropylene polymer of at least 1200 molecular weight (these surfactants are generally described as poloxamers; specific examples are described in the Examples below) as poloxamer 407 and poloxamer 388,
soap powder, and
mixtures thereof.

Examples of suitable coating substances include waxes (both natural and synthetic), silicones, silicone glycol copolymers and polydimethylsiloxanes at molecular weights from between about 700 centistokes (cs) and several million cs. (Specific examples are described in the Examples below including PDMS 2.5 million cs and ULTRAMULSION® 10-2.5.)

Suitable abrasives for use with interproximal dental devices of the present invention, which are designated by the trademark SOFT ABRASIVES®, include dicalcium phosphate (DCP), sodium hexametaphosphate, pumice, aluminum silicate, silica, glass beads, titanium oxide, rice flour, quartz, novaculite, silicon carbide, alumina zirconia, alumina, polishing alumina, calcined aluminum oxide, silicon and zirconium oxide; all of the foregoing at various crystal forms and particle shapes; various hardness including Rockwell 48-50c at various sieve analysis ranging from U.S. sieve #60 to #270; various specific gravity ranges including 2.65 g/cc, 3.20 g/cc, 4.3 g/cc, 3.6 to 3.9 g/cc.

These abrasives are preferably added to saliva-soluble coatings at between about 0.25% and about 20% by weight of the substrate. An alternative method for adding additional abrasive to coated devices is by means of a dusting process where the coated device is passed through a chamber charged with abrasive particles in the air, wherein the abrasive particles coat the coated device as it passes through the dusting chamber.

Suitable abrasives are commercially available from AGSCO Corp., Wheeling, Ill.

Suitable wax coatings are described in U.S. Pat. Nos. 2,667,443; 5,967,153; 5,908,039 and 5,830,495.

The contact coating means used to coat certain of the interproximal dental devices of the present invention comprises: a rotating wheel with a smooth surface to which a controlled level of heated liquid coating substance is continuously applied. Substantially, all of this liquid coating is transferred from the wheel surface to one side of the interproximal device that is released from a package onto said coated wheel and eventually taken up by a driven winding means, generally in a direction counter to the rotation of said wheel, and at a controlled speed. Said device tangentially contacts the coated wheel with minimum contact pressure at the coating transfer area of the wheel which comprises an arc of less than about 30° of said wheel. The contact between one side of said device and the coating on the wheel is sufficient to lift substantially all of the heated liquid coating from the surface of said wheel onto the device.

Shortly after the liquid coating has been transferred to one side of said device, the coated device is passed through a coating equalizing port where a portion of the liquid coating substance is continuously transferred from the coated side to the uncoated side of said device, such that upon exiting the coating equalizing port, both sides of the device are coated at about equal levels with said liquid coating.

The coated device is then cooled and taken up onto a package using standard take-up winding means. The wound coated device, having been cooled, is capable of being wound into a package by the take-up winder with minimum flaking and/or fracturing off of the saliva soluble coating. This take-up winding is carried out with minimum folding or bending of the coated device on itself.

When unwound from the package, the contact coated interproximal dental device remains substantially intact with minimal loss and/or damage to the coating occurring during bobbin winding and with no bending or fibrillating of the coated device.

It will be obvious to one skilled in the art that contact coating methodology allows for coating formulations of a wide variety of viscosities, hydrophobic/hydrophilic properties, solid content and emulsion properties.

Sialagogue coatings described in Formulas A-D set out below are compression coated on texturized multifilament dental flosses of the invention, as detailed in Examples 1 through 4 below.

TABLE 1
Emulsion/Sialagogue Containing Saliva Soluble Coatings
suitable for coating all types of interproximal devices

| | Percentage |
|---|---|
| | (%) by wt. |
| Formula A: | |
| Jambu Oleoresin | 0.1 |
| poloxamer 407 | 44.3 |
| Polydimtheylsiloxane | 17.6 |
| (Dow Corning 1500) | |
| Dicalcium phosphate | 13.3 |
| Carrageenan | 13.3 |
| Flavor | 8.9 |
| Saccharin | 2.2 |
| EDTA | 0.2 |
| Propyl Gallate | 0.1 |
| | 100.0 |
| Formula B: | |
| zingerone | 0.0001 |
| poloxamer 407 | 62 |
| Hydrogenated Vegetable Oil | 20 |
| Sodium Bicarbonate | 18 |
| | 100 |
| Formula C: | |
| capsaicin | 0.0001 |
| poloxamer 407 | 91.74 |
| Sodium Fluoride | 1.6 |
| Cetylpyridium Chloride | 0.6 |
| Domiphen Bromide | 0.06 |
| Hydrogenated Vegetable Oil | 5.0 |
| Carrageenan | 1.0 |
| | 100 |
| Formula D: | |
| Eugenol | 0.001 |
| poloxamer 407 | 87.1 |
| Sorbitol | 10.5 |
| Sodium Fluoride | 1.7 |
| Cetylpyridinium Chloride | 0.63 |
| Domiphen Bromide | 0.07 |
| | 100 |

Examples 1 through 4 include various multifilament dental devices coated with emulsion/sialagogue mixtures of the invention as described in Table 1 above.

### Example 1

When Formula A was introduced into the textured dental floss described above at 1.0 twists per inch (tpi) as per the general process described in U.S. Pat. No. 4,911,927 where the loading tank was held at 90° C., the compression rollers at 90° C., the slub gap at 0.018″, the line speed was 1.6 yd/sec, the resultant load in was 100 mg/yd.

Under the same process conditions standard untextured nylon at 1.5 tpi loaded 50 mg/yd of Formula A. When this standard untextured dental floss was processed with different process conditions, i.e., tank temperature 80° C., slub gap (0.014), line speed 2.0 yd/sec, roller speed 2.4 yd/sec, and line tension 80 to 100 g, the resultant floss load of Formula A was only 40 mg/hg.

### Example 2

When Formula B was introduced into standard nylon floss at 1.5 tpi as well into as textured dental flosses of the invention at 1.0, 1.5 and 2.5 tpi under various processing conditions, the influence of texturing and twist on the load of the chemotherapeutic preparation was not evident.

In this example, the untextured floss processed with the loading tank and rollers at 90° C., the slub gap at 0.018. The line speed and roller speeds were 2.0 and 2.4 yd/sec, respectively. The line tension was 30-100 g. The load was 34 mg/yd.

Textured yarn at 1.0 and 2.5 tpi run at the very same process settings loaded 82 and 42 mg/yd of Formula B, respectively. Textured yarn of 1.5 tpi run at a slightly lower temperature, tank and roller 80° C., and a slightly lower line speed, 1.7 yd/sec, with a roller speed of 2.0 yd/sec and a lower line tension, 40-60 g, produced a load of 75 mg mg/yd of Formula B.

### Example 3

When Formula C was introduced at 1.5 tpi under the following process conditions: Tank 99° C., roller 80° C., gap 0.018″, line speed 1.7 yd/sec, roller speed 2.1 yd/sec, and line tension 80-100 g, the load of Formula C into this texturized dental floss was 64 mg/yd.

### Example 4

When Formula D was introduced into textured floss of the invention at 1.6 tpi and into standard floss at 1.6 tpi under the same process conditions, with tank and roller temperatures 97° C. and 80° C., respectively, gap 0.018″, line speed and roller speed, 1.7 and 1.9 yd/sec respectively, and line tension 80-100 g, the influence of texturing on the load of chemotherapeutic agents into the floss of the present invention was dramatically demonstrated, i.e. 80 mg/yd for textured floss vs. 34 mg/yd for standard floss.

Examples 5 through 22 below illustrate various saliva soluble base coatings for monofilament dental devices, where the base coating contains various emulsion/sialagogue mixtures.

TABLE 2
Illustrative Examples of Substantially Crystal-Free, Flake-Free Base Coatings Containing an Emulsion,
Flavorant and at least one Sialagogue, Suitable for Various Monofilament Interproximal Dental Tapes

| | EXAMPLE | | | | | | | | |
|---|---|---|---|---|---|---|---|---|---|
| Ingredient | 5 | 6 | 7 | 8 | 9 | 10 | 11 | 12 | 13 |
| ULTRAMULSION 10-2.5 | 57.1 | 54.8 | 52.3 | 50.8 | 50.8 | 50.8 | 58.8 | 60.8 | |
| Emsorb 2726 | 12.5 | 7.5 | 12.5 | 9 | 5 | 3 | 3 | 0 | 3 |
| Stearyl Alcohol | 9.2 | 10.5 | 8 | 7 | 11 | 13 | 15 | 16 | 15 |
| Insoluble Saccharin | 1.8 | 1.8 | 1.8 | 1.8 | 1.8 | 1.8 | 1.8 | 1.8 | 1.8 |
| Propyl gallate | 0.1 | 0.1 | 0.1 | 0.1 | 0.1 | 0.1 | 0.1 | 0.1 | 0.1 |
| Spicemint Flavor | 4 | 4 | 4 | 4 | 4 | 4 | 4 | 4 | 5 |
| Vanilla Mint Flavor | 4 | 4 | 4 | 4 | 4 | 4 | 4 | 4 | 5 |
| Tetrasodiumpyrophosphate | 8 | 14 | 14 | 10 | 10 | 10 | 10 | 10 | 10 |
| Dicalcium phosphate | | | | | | | | | |
| Microcrystalline | | | | 10 | 10 | 10 | 0 | 0 | 0 |
| Wax ML 445 | | | | | | | | | |
| Jambu oleoresin | 0.05 | 0.05 | 0.1 | 0.1 | | | | | 0.1 |
| Capsaicin | 0.05 | 0.05 | 0.025 | 0.025 | 0.025 | 0.025 | 0.025 | 0.025 | |
| Zingerone | 0.0001 | 0.0001 | | | | | | | 0.0001 |
| Piperine | 2 ppm | 2 ppm | | | | | | | 2 ppm |
| | EXAMPLE | | | | | | | | |
| Ingredient | 14 | 15 | 16 | 17 | 18 | 19 | 20 | 21 | 22 |
| ULTRAMULSION 10-2.5 | 60.1 | 55.1 | 51.1 | 60.1 | | 61.1 | 61.1 | 53.1 | 57.1 |
| Emsorb 2726 | 3 | 3 | 3 | 3 | 3 | 4 | 3 | 3 | 3 |
| Stearyl Alcohol | 15 | 15 | 15 | 15 | 15 | 10 | 8 | 15 | 15 |
| Insoluble Saccharin | 1.8 | 1.8 | 1.8 | 1.8 | 1.8 | 1.8 | 1.8 | 1.8 | 1.8 |
| Propyl gallate | 0.1 | 0.1 | 0.1 | 0.1 | 0.1 | 0.1 | 0.1 | 0.1 | 0.1 |
| Spicemint Flavor | 5 | 5 | 5 | 5 | 5 | 5 | 5 | 5 | 5 |
| Vanilla Mint Flavor | 5 | 5 | 5 | 5 | 5 | 5 | 5 | 5 | 5 |
| Tetrasodiumpyrophosphate | | 10 | 14 | 4 | | 6 | 6 | 10 | 6 |
| Dicalcium phosphate | 10 | | | | | | | | |
| Microcrystalline | 0 | 5 | 5 | | 0 | 7 | 10 | 7 | 7 |
| Wax ML 445 | | | | | | | | | |
| Jambu oleoresin | 0.1 | 0.05 | 0.05 | | | | | 0.025 | 0.025 |
| Capsaicin | | | | | 0.05 | 0.025 | 0.025 | | |
| Zingerone | 0.0002 | 0.0002 | 0.0002 | | | 0.0001 | 0.0001 | | |
| Piperine | 2 ppm | 1 ppm | 1 ppm | 2 ppm | 2 ppm | 1 ppm | 1 ppm | | |

Various monofilament interproximal dental tapes were coated with coatings such as described in Table 2 as follows:

### Example 23

Expanded PTFE tape, 1100 denier, was coated using the compression roller method with a formula consisting of ULTRAMULSION® 57.1%; Emsorb 2726, 3.0%, stearyl alcohol, 9.2%; insoluble saccharin, 1.8%; propyl gallate, 0.1%; spicemint flavor, 4.0%; vanillamint flavor, 4.0%; tetrasodium pyrophosphate, 8.0%; jambu oleoresin, 0.05%, capsaicin, 0.05%. The tape was coated with a load of 68 mG/yd.

### Example 24

High density polyethylene tape, 478 denier, was coated using a kiss wheel method using the formula: ULTRAMULSION 10/2.5, 60.1; emsorb 2726, 3.0%; stearyl alcohol, 15.0%; insoluble saccharin, 1.8%; propyl gallate, 0.1%; spicemint flavor, 5.0%; vanillamint flavor 5.0%; dicalcium phosphate, 10.0%; jambu oleoresin, 0.1%. The tape was coated with a load of 57 mG/yd

### Example 25

Ultra high molecular weight polyethylene tape, with fibrillations, was compression loaded with a formula: ULTRAMULSION 10/2.5, 57.1; emsorb 2726, 3.0%; stearyl alcohol, 15.0%; insoluble saccharin, 1.8%; propyl gallate, 0.1%; spicemint flavor 5.0%; vanillamint flavor, 5.0%; tetrasodium pyrophosphate, 6.0%, microcrystalline wax, 7.0%; jambu oleoresin, 0.025%. The tape was coated with a load of 77 mG/yd.

TABLE 3
Illustrative Examples of Suitable Coated Monofilament Dental Tapes

| | | | | | | | | PROCESSING | | |
|---|---|---|---|---|---|---|---|---|---|---|
| | | | | TAPE COMPOSITION | | | Secondary | CONDITIONS | | |
| | Thermoplastic | | | | Silicone | | Polymer | Melt | Draw | |
| Ex. | Elastomer | | Trade | | Process | TiO₂ | added | Temp | Temp | Draw |
| No. | Type | Manufacturer | Name | Grade | Aid (%) | (%) | Type (%) | ° C. | ° C. | Ratio |
| 26 | PEBA polyester amide | Atofina | PEBAX | 55/33 | 3.5 | 1.8 | PP-4.7 | 260 | 130 | 6.8:1 |
| 27 | ″ | ″ | ″ | ″ | 3.5 | 1.8 | PP-4.7 | 260 | 130 | 6.5:1 |
| | | | | | | | Adflex-5 | | | |
| 28 | ″ | ″ | ″ | ″ | 3.5 | 1.8 | PP-9.7 | 260 | 130 | 6.8:1 |
| 29 | PEBA polyester amide | Atofina | PEBAX | 63/33 | 0 | 0 | 0 | 260 | 135 | 6:1 |
| 30 | ″ | ″ | ″ | ″ | 0 | 1.8 | PP-1.2 | 260 | 135 | 6.3:1 |
| 31 | ″ | ″ | ″ | ″ | 3.5 | 1.8 | PP-47 | 260 | 135 | 6.2:1 |
| 32 | ″ | ″ | ″ | ″ | 3.5 | 1.8 | PP-4.7 | 260 | 135 | 6.2:1 |
| | | | | | | | Adflex-5 | | | |
| 33 | ″ | ″ | ″ | ″ | 3.5 | 1.8 | PP-9.7 | 260 | 135 | 6.2:1 |
| 34 | ″ | ″ | ″ | ″ | 3.5 | 1.8 | PP-4.7 | 260 | 135 | 6.2:1 |
| | | | | | | | Nylon 11-5 | | | |
| 35 | TPE polyether ester | DuPont | Hytrel | 6359FG | 2.3 | 1.0 | 0 | 225 | 130 | 5:1 |
| 36 | ″ | ″ | ″ | ″ | 3.5 | 1.8 | PP-4.7 | 225 | 140 | 5.7:1 |
| 37 | TPE-E polyether ester | DSM | Arnitel | PM581 | 0 | 0 | 0 | 235 | 140 | 4.3:1 |
| 38 | ″ | ″ | ″ | ″ | 0 | 1.8 | PP-1.2 | 240 | 115 | 4.3:1 |
| 39 | ″ | ″ | ″ | ″ | 3 | 0 | PBT-5 | 235 | 140 | 4.3:1 |
| 40 | ″ | ″ | ″ | ″ | 0 | 0 | PBT-5 | 235 | 140 | 4.3:1 |
| 41 | ″ | ″ | ″ | ″ | 0 | 1.8 | PP-1.2 | 235 | 140 | 4.3:1 |
| | | | | | | | PBT-5 | | | |

| | PROPERTIES | | | | | | UTILITY FACTORS | | | Coating |
|---|---|---|---|---|---|---|---|---|---|---|
| | Break | Elongation | | | | | | Tape | | from |
| | Strength | to | | | DIMENSIONS | | | Flex | | Examples |
| | in | Break | Elastic | | Width | | Gentleness | Twist | Hardness | of Table 2 |
| Ex. No. | Newtons | (%) | Limit | Decitex | (mm) | Thick (mm) | Perception | Index | Shore D | (in mg/yd) |
| 26 | 30 | 26 | 0 | 750 | 1.30 | 0.063 | 6 | 4 | 37 | Ex 5 (62) |
| 27 | 27 | 18 | 0 | 760 | 1.30 | 0.063 | 6 | 4 | 37 | Ex 6 (58) |
| 28 | 26 | 19 | 0 | 760 | 1.30 | 0.063 | 6 | 4 | 37 | Ex 7 (54) |
| 29 | 30 | 15 | 0 | 805 | 1.44 | 0.065 | 5.5 | 4 | 36 | Ex 8 (52) |
| 30 | 32.36 | 13 | 0 | 800 | 1.41 | 0.067 | 5.5 | 4 | 36 | Ex 9 (50) |
| 31 | 33.47 | 17 | 0 | 860 | 1.36 | 0.066 | 5.5 | 4 | 36 | Ex 10 (54) |
| 32 | 25.94 | 14 | 0 | 810 | 1.32 | 0.078 | 5.5 | 4 | 36 | Ex 11 (32) |
| 33 | 29.46 | 14 | 0 | 780 | 1.34 | 0.069 | 5.5 | 4 | 36 | Ex 12 (44) |
| 34 | 30.63 | 13 | 0 | 810 | 1.30 | 0.065 | 5.5 | 4 | 36 | Ex 13 (48) |
| 35 | 20 | 20 | 15 | 1400 | 1.70 | 0.070 | 7 | 3 | 33 | Ex 14 (48) |
| 36 | 24 | 14 | 10 | 1230 | 1.70 | 0.070 | 7 | 3 | 33 | Ex 15 (56) |
| 37 | 18 | 13 | 10 | 1500 | 1.63 | 0.084 | 7 | 3 | 33 | Ex 16 (60) |
| 38 | 19 | 14 | 5 | 1634 | 1.64 | 0.085 | 7 | 3 | 33 | Ex 17 (52) |
| 39 | 19 | 10 | 2 | 1580 | 1.68 | 0.079 | 7 | 3 | 33 | Ex 18 (38) |
| 40 | 18 | 12 | 3 | 1500 | 1.70 | 0.086 | 7 | 3 | 33 | Ex 19 (36) |
| 41 | 21 | 15 | 4 | 1575 | 1.77 | 0.083 | 7 | 3 | 33 | Ex 20 (32) |

TABLE 4
Illustrative Examples of Suitable Coated Monofilament Dental Tapes

| | | | | | | | | PROCESSING | | |
|---|---|---|---|---|---|---|---|---|---|---|
| | | | | TAPE COMPOSITION | | | Secondary | CONDITIONS | | |
| | Thermoplastic | | | | Silicone | | Polymer | Melt | Draw | |
| Ex. | Elastomer | | Trade | | Process | TiO₂ | added | Temp | Temp | Draw |
| No. | Type | Manufacturer | Name | Grade | Aid (%) | (%) | Type (%) | ° C. | ° C. | Ratio |
| 42 | TPE-E polyether ester | DSM | Arnitel | EM550 | 0 | 0 | 0 | 240 | 140 | 4.3:1 |
| 43 | ″ | ″ | ″ | ″ | 0 | 1.8 | PP-1.2 | 240 | 115 | 6:1 |
| 44 | ″ | ″ | ″ | ″ | 0 | 1.8 | PP-6.2 | 240 | 140 | 5.6:1 |
| 45 | ″ | ″ | ″ | ″ | 0 | 0 | PBT-5 | 240 | 145 | 5.9:1 |
| 46 | TPE-E polyester ester | OSM | Arnitel | EM630 | 0 | 0 | 0 | 235 | 150 | 4.5:1 |
| 47 | ″ | ″ | ″ | ″ | 0 | 1.8 | PP-1.2 | 235 | 150 | 4.7:1 |
| 48 | ″ | ″ | ″ | ″ | 0 | 1.8 | PP-1.2 | 235 | 150 | 4.6:1 |
| | | | | | | | Adflex-5 | | | |
| 49 | ″ | ″ | ″ | ″ | 0 | 1.8 | PP-6.2 | 235 | 150 | 4.6:1 |
| 50 | ″ | ″ | ″ | ″ | 0 | 0 | PBT-5 | 235 | 150 | 4.6:1 |
| 51 | TPE-E polyester ester | DSM | Arnitel | UM552 | 0 | 0 | 0 | 240 | 150 | 6.6:1 |
| 52 | ″ | ″ | ″ | ″ | 0 | 0 | 0 | 230 | 150 | 5.6:1 |
| 53 | ″ | ″ | ″ | ″ | 0 | 1.8 | PP-1.2 | 240 | 150 | 6.3:1 |
| 54 | ″ | ″ | ″ | ″ | 0 | 0 | Adflex-5 | 230 | 140 | 5.6:1 |
| 55 | ″ | ″ | ″ | ″ | 0 | 1.5 | PP-1.2 | 230 | 145 | 5.7:1 |
| | | | | | | | PBT-5 | | | |
| 56 | ″ | ″ | ″ | ″ | 0 | 0 | PBT-5 | 230 | 245 | 5.3:1 |
| 57 | EPDM TPV | Monteil | Adflex | Q100F | 0 | 0 | PP-20 | 240 | 130 | 4.5:1 |
| 58 | ″ | ″ | ″ | ″ | 3.5 | 1.8 | PP-24.7 | 240 | 130 | 4.5:1 |
| 59 | ″ | ″ | ″ | ″ | 0 | 3 | PP-30 | 240 | 130 | 4.7:1 |
| 60 | ″ | ″ | ″ | ″ | 0 | 3 | PP-34.7 | 240 | 130 | 4.7:1 |
| 61 | ″ | ″ | ″ | ″ | 0 | 3 | PP-40 | 240 | 130 | 4.7:1 |

| | PROPERTIES | | | | | | UTILITY FACTORS | | | Coating |
|---|---|---|---|---|---|---|---|---|---|---|
| | Break | Elongation | | | | | | Tape | | From |
| | Strength | To | | | DIMENSIONS | | | Flex | | Examples |
| Ex. | in | Break | Elastic | | Width | | Gentleness | Twist | Hardness | of Table 2 |
| No. | Newtons | (%) | Limit | Decitex | (mm) | Thick (mm) | Perception | Index | Shore D | (in mg/yd) |
| 42 | 23 | 25 | 7 | 1800 | 1.95 | 0.096 | 7 | 3 | 33 | Ex 21 (30) |
| 43 | 27 | 11 | 5 | 1050 | 1.47 | 0.071 | 7 | 3 | 33 | Ex 22 (36) |
| 44 | 26 | 17 | 5 | 1216 | 1.45 | 0.071 | 7 | 3 | 33 | Ex 5 (40) |
| 45 | 28 | 145 | 5 | 1220 | 1.55 | 0.074 | 7 | 3 | 33 | Ex 6 (42) |
| 46 | 18 | 12 | 4 | 1090 | 1.44 | 0.067 | 7 | 3 | 33 | Ex 7 (48) |
| 47 | 17 | 11 | 4 | 1130 | 1.50 | 0.068 | 7 | 3 | 33 | Ex 8 (60) |
| 48 | 18 | 10 | 7 | 961 | 1.35 | 0.065 | 7 | 3 | 33 | Ex 9 (42) |
| 49 | 14 | 30 | 10 | 965 | 1.24 | 0.073 | 7 | 3 | 33 | Ex 10 (50) |
| 50 | 20 | 12 | 5 | 1018 | 1.39 | 0.069 | 7 | 3 | 33 | Ex 11 (62) |
| 51 | 32 | 12 | 8 | 1300 | 1.49 | 0.070 | 7.5 | 3.5 | 31 | Ex 12 (70) |
| 52 | 26 | 15 | 8 | 1090 | 1.40 | 0.070 | 7.5 | 3.5 | 31 | Ex 13 (46) |
| 53 | 29 | 16 | 8 | 1150 | 1.46 | 0.070 | 7.5 | 3.5 | 31 | Ex 14 (52) |
| 54 | 30 | 16 | 10 | 1233 | 1.48 | 0.069 | 7.5 | 3.5 | 31 | Ex 15 (42) |
| 55 | 22 | 19 | 10 | 1108 | 1.53 | 0.067 | 7.5 | 3.5 | 31 | Ex 16 (44) |
| 56 | 24 | 14 | 8 | 1143 | 1.48 | 0.064 | 7.5 | 3.5 | 31 | Ex 17 (48) |
| 57 | 26 | 20 | 0 | 910 | 1.60 | 0.064 | 5.5 | NT | NT | Ex 18 (52) |
| 58 | 25 | 24 | 0 | 940 | 1.59 | 0.064 | 5.5 | NT | NT | Ex 19 (50) |
| 59 | 28 | 20 | 0 | 870 | 1.58 | 0.064 | 5.5 | NT | NT | Ex 20 (52) |
| 60 | 27 | 23 | 0 | 880 | 1.58 | 0.060 | 5.5 | NT | NT | Ex 21 (40) |
| 61 | 35 | 18 | 0 | 720 | 1.44 | 0.063 | 5 | NT | NT | Ex 22 (42) |

TABLE 5
Illustrative Examples of Suitable Coated Monofilament Dental Tapes

| | | | | | | | | PROCESSING | | |
|---|---|---|---|---|---|---|---|---|---|---|
| | | | | TAPE COMPOSITION | | | Secondary | CONDITIONS | | |
| | Thermoplastic | | | | Silicone | | Polymer | Melt | Draw | |
| Ex. | Elastomer | | Trade | | Process | TiO₂ | added | Temp | Temp | Draw |
| No. | Type | Manufacturer | Name | Grade | Aid (%) | (%) | Type (%) | ° C. | ° C. | Ratio |
| 62 | PEBA polyester amide | Atofina | PEBAX | 55133 | 0 | 1.8 | PP-1.2 | 260 | 130 | 6.8:1 |
| 63 | ″ | ″ | ″ | ″ | 3.5 | 1.8 | PP-4.7 | 260 | 130 | 7:1 |
| | | | | | | | EMA-3 | | | |
| 64 | ″ | ″ | ″ | ″ | 3.5 | 1.8 | PP-4.7 | 260 | 130 | 6.8:1 |
| 65 | PEBA | Atofina | PEBAX | 63/33 | 3.5 | 1.8 | PP-4.7 | 260 | 135 | 6.5:1 |
| | | | | | | | EMA-3 | | | |
| 66 | ″ | ″ | ″ | ″ | 0 | 0 | Nylon 11-5 | 260 | 135 | 6.2:1 |
| 67 | TPE-E polyether ester | DSM | Arnitel | PM581 | 3 | 0 | 0 | 235 | 140 | 5:1 |
| 68 | TPE-E polyether ester | DSM | Arnitel | EM550 | 3 | 0 | 0 | 240 | 140 | 6:1 |
| 69 | ″ | ″ | ″ | ″ | 3 | 1.8 | PP-1.2 | 240 | 140 | 6:1 |
| | | | | | | | EMA-3 | | | |
| 70 | TPE-E polyether ester | DSM | Arnitel | UM552 | 3 | 1.8 | PP-1.2 | 240 | 150 | 6:1 |

| | | | | | | | | | | Coating |
|---|---|---|---|---|---|---|---|---|---|---|
| | PROPERTIES | | | | | | UTILITY FACTORS | | | from |
| | Break | Elongation | | | | | | Tape | | Examples |
| | Strength | to | | | | | | Flex | | of |
| Ex. | in | Break | Elastic | | DIMENSIONS | | Gentleness | Twist | Hardness | Table 2 |
| No. | Newtons | (%) | Limit | Decitex | Width (mm) | Thick (mm) | Perception | Index | Shore D | (in mg/yd) |
| 62 | 28 | 24 | 0 | 775 | 1.30 | 0.063 | 6 | 4 | 37 | Ex 5 (40) |
| 63 | 28 | 30 | 3 | 750 | 1.30 | 0.063 | 8 | 4 | 37 | Ex 6 (52) |
| 64 | 29 | 24 | 0 | 800 | 1.35 | 0.070 | 6 | 4 | 37 | Ex 7 (42) |
| 65 | 31 | 20 | 3 | 800 | 1.40 | 0.065 | 5.5 | 4 | 36 | Ex 8 (60) |
| 66 | 28 | 14 | 0 | 800 | 1.30 | 0.065 | 5.5 | 4 | 36 | Ex 9 (48) |
| 67 | 22 | 16 | 7 | 1400 | 1.60 | 0.079 | 7 | 3 | 33 | Ex 10 (42) |
| 68 | 25 | 20 | 7 | 800 | 1.30 | 0.060 | 7 | 3 | 33 | Ex 11 (60) |
| 69 | 27 | 15 | 5 | 850 | 1.35 | 0.065 | 7 | 3 | 33 | Ex 12 (42) |
| 70 | 27 | 17 | 10 | 1100 | 1.47 | 0.069 | 7.5 | 3 | 33 | Ex 13 (60) |

TABLE 6
Examples of Suitable Monofilament Dental Tapes

| | | | | | | | | PROCESSING | | |
|---|---|---|---|---|---|---|---|---|---|---|
| | | | | TAPE COMPOSITION | | | Secondary | CONDITIONS | | |
| | Thermoplastic | | | | Silicone | | Polymer | Melt | Draw | |
| Ex. | Elastomer | | Trade | | Process | TiO₂ | added | Temp | Temp | Draw |
| No. | Type | Manufacturer | Name | Grade | Aid (%) | (%) | Type (%) | ° C. | ° C. | Ratio |
| 71 | Styrenics SEBS | Alphagary | Evoprene | G978 | 0 | 1.8 | PP-1.2 | 200 | 100 | 7:1 |
| 72 | ″ | ″ | ″ | ″ | 3 | 1.8 | PP-1.2 | 200 | 100 | 7:1 |
| 73 | ″ | ″ | ″ | ″ | 0 | 1.8 | PP-1.2 | 200 | 100 | 7.2:1 |
| | | | | | | | EMA-3 | | | |
| 74 | ″ | ″ | ″ | ″ | 3.5 | 1.8 | PP-9.7 | 200 | 100 | 7:1 |
| 75 | ″ | ″ | ″ | ″ | 3.5 | 1.8 | PP-9.7 | 200 | 100 | 7:1 |
| | | | | | | | PS-5 | | | |
| 76 | TPU 90 AEN | Dow | Pelethane | 2103 | 0 | 1.8 | PP-1.2 | 230 | 120 | 7:1 |
| 77 | ″ | ″ | ″ | ″ | 3 | 1.8 | PP-1.2 | 230 | 120 | 6:1 |
| 78 | ″ | ″ | ″ | ″ | 0 | 1.8 | PP-1.2 | 230 | 120 | 6:1 |
| | | | | | | | EMA-3 | | | |
| 79 | ″ | ″ | ″ | ″ | 3.5 | 1.8 | PP-9.7 | 230 | 120 | 5:1 |
| 80 | TPV | DSM | Sarlink | 4149D | 0 | 1.8 | PP-1.2 | 220 | 105 | 4.5:1 |
| 81 | ″ | ″ | ″ | ″ | 3 | 1.8 | PP-1.2 | 220 | 105 | 5:1 |
| 82 | ″ | ″ | ″ | ″ | 0 | 1.8 | PP-1.2 | 220 | 105 | 4.8:1 |
| | | | | | | | EMA-3 | | | |
| 83 | ″ | ″ | ″ | ″ | 3 | 1.8 | PP-6.2 | 220 | 105 | 4.2:1 |

| | | | | | | | | | | Coating |
|---|---|---|---|---|---|---|---|---|---|---|
| | PROPERTIES | | | | | | UTILITY FACTORS | | | from |
| | Break | Elongation | | | | | | Tape | | Examples |
| | Strength | to | | | DIMENSIONS | | | Flex | | of |
| | in | Break | Elastic | | Width | Thick | Gentleness | Twist | Hardness | Table 2 |
| Ex. No. | Newtons | (%) | Limit | Decitex | (mm) | (mm) | Perception | Index | Shore D | (in mg/yd) |
| 71 | 19 | 30 | 10 | 1100 | 1.30 | 0.060 | 6 | 4 | 37 | Ex 14 (52) |
| 72 | 20 | 35 | 12 | 1100 | 1.30 | 0.060 | 6 | 4 | 37 | Ex 15 (56) |
| 73 | 17 | 32 | 12 | 1100 | 1.30 | 0.060 | 6 | 4 | 37 | Ex 16 (62) |
| 74 | 14 | 20 | 7 | 1100 | 1.30 | 0.060 | 8 | 4 | 37 | Ex 17 (64) |
| 75 | 22 | 28 | 8 | 1100 | 1.30 | 0.060 | 6 | 4 | 37 | Ex 18 (70) |
| 76 | 32 | 15 | 5 | 1200 | 1.40 | 0.068 | 7 | 3 | 33 | Ex 19 (72) |
| 77 | 30 | 17 | 6 | 1200 | 1.40 | 0.068 | 7 | 3 | 33 | Ex 20 (42) |
| 78 | 26 | 16 | 6 | 1200 | 1.40 | 0.068 | 7 | 3 | 33 | Ex 21 (40) |
| 79 | 22 | 10 | 2 | 1300 | 1.45 | 0.070 | 7 | 3 | 33 | Ex 22 (52) |
| 80 | 20 | 20 | 5 | 1400 | 1.45 | 0.072 | 6 | 4 | 37 | Ex 5 (40) |
| 81 | 22 | 35 | 7 | 1300 | 1.40 | 0.070 | 6 | 4 | 37 | Ex 6 (62) |
| 82 | 19 | 20 | 5 | 1350 | 1.48 | 0.075 | 6 | 4 | 37 | Ex 7 (64) |
| 83 | 15 | 20 | 5 | 1450 | 1.48 | 0.075 | 6 | 4 | 37 | Ex 8 (52) |

### Example 84

A coating emulsion was prepared by melting 67 grams of poloxamer 338, adding 6.7 grams of polydimethylsiloxane (12.500 CS) and stirring with an overhead stirrer at 88 degrees C. The melt was homogenized with a rotor/stator overhead mixer until creamy in consistency. Eight grams of peppermint oil was added with stirring and 2.3 grams of acid saccharin was added. A flavor component, 0.4 grams, containing spilanthol, IFF SN554170, was added and mixed for 20 minutes. Dental picks were dipped into the coating emulsion and allowed to come to room temperature. Use of the dental picks between the teeth produced a warm sensation and increased saliva flow.

### Example 85

A coating emulsion was prepared by melting 47.1 grams of poloxamer 407 and stirring in 4.6 grams of polydimethylsiloxane, 1000 CS, with heating to 85 degrees centigrade. An overhead stirrer was employed to achieve a creamy emulsion. Three grams of Emsorb 2726-C was added along with 15 grams of stearyl alcohol. After this was fully emulsified, 15 grams of Microwax ML-445 was added with continued heating and stirring. Carbowax 8000, 1.4 grams, was added along with 6 grams of dicalcium phosphate. Further addition of EDTA, 0.2 grams, propyl gallate, 0.1 grams, menthol, 2 grams, and 6 grams of Spicemint flavor was completed with stirring. The sialagogue containing flavor, IFF SN554170, was added with stirring. After mixing for 20 minutes, proxy brushes from retail distributors, such as Butler, Oral-B and Perident, were pushed through a hole in a thin stainless steel plate sized to give a residual level of from 10 to 30 milligrams of coating emulsion. These coated proxy brushes were then used between the teeth to clean debris and leave behind a fresh, tingling feeling.

### Example 86

A coating was prepared using a vessel fitted with an overhead stirrer. ULTRAMULSION® 10/2.5, 8.14 grams, was stirred into 32.56 grams of water over ten minutes. 20 grams of sorbitol-70 was added to the stirring suspension over .1 minute. Sodium saccharin, 3.3 grams was added to the suspension with stirring along with sodium benzoate, 0.1 grams. Ethanol, 20 grams, was added to another vessel along with vanillamint flavor, 15 grams; cooling agent, 0.4 grams and jambu oleoresin, 0.5 grams. The ethanol solution of flavors and sensate agents was stirred into the above suspension over 2 minutes to give a pale yellow opaque suspension. The tips of flosser head units for the ACCESS brand of flosser were dipped partially into the suspension and set aside for 24 hours to dry. The flosser heads were coated with 12 milligrams of coating. The flosser heads were dipped into finely powdered dicalcium phosphate to reduce tackiness.

### Example 87

A vessel was fitted with an overhead stirrer and water, 36.63 grams, was added. ULTRAMULSION® 20/2.5, 4.07 grams, was added over ten minutes. Sorbitol-70, 20.0 grams, was added over 1 minute. Sodium saccharin, 3.3 grams, were added along with sodium benzoate, 0.1 grams with stirring. To another vessel was added 20 grams of ethanol, 15 grams of vanillamint flavor, cooling agent 0.4 grams, jambu oleoresin, 0.5 grams. The contents were added to the stirred suspension to give a light yellow suspension. Plackers Twinline flossers were dipped ⅛^{th} inch into the suspension and then dried for 24 hours. The tips were dipped into dicalcium phosphate to reduce tackiness.

### Example 88

A vessel was fitted with an overhead stirrer and water, 36.63 grams, was added. ULTRAMULSION® 20/2.5, 4.07 grams, was added over ten minutes. Sorbitol-70, 20.0 grams, was added over 1 minute. Sodium saccharin, 3.3 grams, were added along with sodium benzoate, 0.1 grams with stirring. To another vessel was added 20 grams of ethanol, 15 grams of vanillamint flavor, cooling agent 0.4 grams, jambu oleoresin, 0.5 grams. The contents were added to the stirred suspension to give a light yellow suspension. The Butler Gum brand rubber tipped massager was coated with the suspension and dried overnight.

The following formulas can be coated on HDPE tape and non-textured multifilament yarn as previously described using the contact coating method:

| Ingredient | % | Load Level Tape | Load Level Yarn |
|---|---|---|---|
| Example 89: | | | |
| Microcrystalline Wax | 84.0 | 40 mg/yd | 32 mg/yd |
| Natural Beeswax | 10.0 | | |
| Saccharin | 2.0 | | |
| Menthol | 3.0 | | |
| Jambu Oleoresin | 1.0 | | |
| Example 90: | | | |
| Lipowax ® | 79.0 | 53 mg/yd | 44 mg/yd |
| Natural Beeswax | 10.0 | | |
| Saccharin | 2.3 | | |
| Vanillamint Flavor | 6.0 | | |
| Menthol | 2.5 | | |
| Jambu Oleoresin | 0.2 | | |
| Example 91: | | | |
| PEG 8000 | 84.0 | 45 mg/yd | 39 mg/yd |
| Polysorbate 80 | 5.0 | | |
| Saccharin | 2.0 | | |
| Vanillamint Flavor | 6.0 | | |
| Menthol | 2.5 | | |
| Jambu Oleoresin | 0.5 | | |

TABLE 8
Comparison of commercial coated monofilament tapes with compression coated
ParaFloss ™ containing a sialagogue

| Brand of | | | | | | |
|---|---|---|---|---|---|---|
| Monofilament | | Comfort | | easySLIDE | | ParaFloss ™ |
| Tape | Glide ® | Plus ® | easySLIDE ® | Pro ™ | Fibaclean ® | Dental Tape |
| Chemical | PTFE | PTFE | PTFE | Ultra high | PEBAX ® | HDPE |
| composition | | | | molecular | | (See |
| of | | | | weight | | Example 1) |
| monofilament | | | | Polyethylene | | |
| | | | | tape | | |
| Method of | Extruded | Extruded | Extruded | Compacted/ | Extruded | Extruded/ |
| manufacturing | | | | fibrillated | | blown, |
| | | | | | | subsequently |
| | | | | | | slit and |
| | | | | | | drawn |
| Avg. Width | 47 | 78 | 53 | 62 | 51 | 68 |
| in mils | | | | | | |
| Avg. | 2.4 | 2.7 | 2.2 | 1.3 | 2.4 | 1.3 |
| Thickness | | | | | | |
| in mils | | | | | | |
| Avg. Denier | 1200 | 1000 | 1500 | 420 | 740 | 480 |
| Percent | 37 | 29 | 15 | 7 | 31 | 10 |
| elongation | | | | | | |
| Avg. Break | 5 | 4.2 | 5.2 | 9.7 | 7.0 | 7.0 |
| Strength | | | | | | |
| Avg. Tenacity | 1.9 | 1.5 | 1.6 | 10.4 | 4.3 | 6.6 |
| g/den | | | | | | |
| Type of | Wax | Wax (8) | Wax (1.3) | Compression | Compression | Contact |
| Coating | (6.1) | | | (65) | (55) | coated (40) |
| Application | | | | | | |
| (in mg/yd) | | | | | | |
| Sialagogue | Jambu | Zingerone | Eugenol | Jambu resin | Capsaicin | Jambu resin |
| (% by wt.) | resin | (0.1) | (0.6) | (0.4) | (0.001) | (0.5) |
| | (0.6) | | | | | |
| Saliva | Insoluble | Insoluble | Insoluble | Soluble | Soluble | Soluble |
| Solubility of | | | | | | |
| coating | | | | | | |
| Hand | medium | good | medium | good | good | good |
| Flavor Impact | 0-1 | 0-1 | 0-1 | 5-6 | 3-4 | 9-10 |
| on a Scale of | | | | | | |
| 0-10 | | | | | | |

TABLE 9
Flavor Impact of Commercial Monofilament Tapes compared to ParaFloss ™,
compression coated with MICRODENT ®/Sialagogue, along with Leading Edge Assessment

| | | | | | | | | | | | | | | | | Flavor |
|---|---|---|---|---|---|---|---|---|---|---|---|---|---|---|---|---|
| | | | Thickness | | | | | | Width | | | | | | | Impact on |
| | Coating | | in mils | | | | | Avg | in mils | | | | | | Leading Edge | 0-10 |
| Product | mg/yd | Denier | 1 | 2 | 3 | 4 | 5 | Thickness | 1 | 2 | 3 | 4 | 5 | Avg. Width | Assessment | scale |
| Comfort Plus ® | 8 | 1000 | 2.4 | 2.7 | 2.2 | 2.2 | 2.3 | 2.36 | 71 | 75 | 79 | 71 | 75 | 74 | Folded - Very gentle | 0-1 |
| GLIDE ® | 6.1 | 1200 | 2.2 | 2.2 | 2.3 | 2.3 | 2.3 | 2.26 | 47 | 46 | 48 | 47 | 47 | 47 | Folded - Gentle | 0-1 |
| easySLIDE ® | 1.3 | 1500 | 1.9 | 1.8 | 2.4 | 2.0 | 2.0 | 2.0 | 55 | 59 | 59 | 55 | 57 | 57 | Folded - Very gentle | 0-1 |
| Fibaclean ™ | 55 | 740 | 2.2 | 2.4 | 2.2 | 2.4 | 2.5 | 2.34 | 63 | 63 | 71 | 67 | 71 | 71 | Extruded edge - | 3-4 |
| | | | | | | | | | | | | | | | Fairly gentle | |
| ParaFloss ™ | 57 | 478 | 1.2 | 1.1 | 1.2 | 1.2 | 1.16 | 1.16 | 75 | 75 | 71 | 71 | 75 | 74 | Rounded - Very gentle | 9-10 |
| 478 denier | | | | | | | | | | | | | | | | |
| coated with | | | | | | | | | | | | | | | | |
| MICRODENT ®/ | | | | | | | | | | | | | | | | |
| Sialagogue | | | | | | | | | | | | | | | | |
| (see Ex. 21) | | | | | | | | | | | | | | | | |
| ParaFloss ™ | 29 | 286 | 1.2 | 1.1 | 1.1 | 1.1 | 1.1 | 1.12 | 39 | 47 | 47 | 43 | 47 | 45 | Rounded - Very gentle | 8-9 |
| 286 denier | | | | | | | | | | | | | | | | |
| coated with | | | | | | | | | | | | | | | | |
| MICRODENT ®/ | | | | | | | | | | | | | | | | |
| Sialagogue | | | | | | | | | | | | | | | | |
| (described in | | | | | | | | | | | | | | | | |
| Table 1 | | | | | | | | | | | | | | | | |
| Formula A) | | | | | | | | | | | | | | | | |

The present invention has been described in detail, including the preferred embodiments thereof. However, it will be appreciated that those skilled in the art, upon consideration of the present disclosure, may make modifications and/or improvements on this invention and still be within the scope of this invention as set forth in the following claims.

What is claimed is:

1. Biofilm therapy dental devices coated with a sialagogue, said devices being suitable for controlling, disrupting and physically removing biofilms while increasing and prolonging saliva flow.

2. Biofilm therapy dental devices coated with a wax/sialagogue mixture, said devices being suitable for controlling, disrupting and physically removing biofilms while increasing and prolonging saliva flow.

3. Biofilm therapy dental devices coated with a sialagogue and surfactant mixture, said devices being suitable for controlling, disrupting and physically removing biofilms while increasing and prolonging saliva flow.

4. Biofilm therapy interproximal dental devices coated with biofilm-responsive saliva soluble coatings comprising an emulsion and at least one sialagogue;
wherein said devices are suitable for:
(a) controlling, disrupting and physically removing biofilms, and
(b) increasing and prolonging saliva flow.

5. Biofilm therapy interproximal dental devices coated with biofilm-responsive saliva soluble coatings comprising a substantially water-free emulsion having a surfactant continuous phase and a polydimethylsiloxane discontinuous phase, where said emulsion contains at least one sialagogue;
wherein said devices are suitable for:
(c) controlling, disrupting and physically removing biofilms, and
(d) increasing and prolonging saliva flow.

6. Biofilm therapy interproximal dental devices, according to claim 4, wherein said dental devices are selected from the group of interproximal dental devices consisting of: multifilament dental flosses, monofilament dental tapes, dental flossers, proxy brushes, dental massagers, dental picks, interdental irrigators and combinations thereof.

7. Biofilm therapy interproximal dental devices, according to claim 5, wherein said surfactant continuous phase is selected from the group of surfactants consisting of nonionic, cationic, anionic, amphoteric surfactants and mixtures thereof.

8. Biofilm therapy interproximal devices, according to claim 5, wherein said polydimethylsiloxane has a viscosity between about 1000 cs and about 10 million cs.

9. Biofilm therapy interproximal dental devices, according to claim 5, wherein said saliva soluble coating is overcoated with a soft abrasive particulate.

10. Biofilm therapy interproximal dental devices, according to claim 3, wherein said sialagogue is selected from the group consisting of synthetic and natural plant extracts, N-Isobutyl-2,6,8-decatrienamide, amides of vegetable origin, Heliopsis longipes, Chiluan Root derivatives, N-isobutylamides from buds of Spilanthes acmella, capsacinia, alkamides of flavoring plants, pepper alkanides, pilocarpine alkanides, ginger alkanides, ginseng parts, Jambo Oleoresin, Zanthoxylom peperitum, saanshaol-1, Saanshoal-II, sanshoamide, isopulegole, 3-(1-menthoxy) propan-1,1-diol, p-menthan-3,8-diol, 6-isopropyl-9-methyl-1,4-dioxaspiro-(4,5)-decane-2-methanol, menthyl succinate, menthyl succinate, trimethyl cyclohexanol, N-ethyl-2-isopropyl-5-methylcyclohexane carboxamide, 3-(1-menthoxy)-2-methyl-propan-1,2-diol, menthone glycerin ketal, menthyl lactate, [1′R,2′S,5′R]-2-(5′methyl-2′-(methylethyl) cyclohexyloxy)ethan-1-ol, [1′R,2′S,5′R]-3-(5′-methyl-2′-(methylethyl)cyclohexyloxy)propan-1-ol, [1′R,2′S,5′R]-4-(5′-methyl-2′-(methylethyl)cyclohexyloxy)butan-1-ol, vanillyl ethyl ether, vanillyl propyl ether, vanillin propylene glycol acetal, ethyl vanillin propylene glycol acetal, gingerol, vanillyl butyl ether, 4-(1-menthoxy-methyl)-2-phenyl-1,3-dioxolane, 4-(1-menthoxy-methyl)-2-(3′,4′-dihydrox-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′4′-methylenedioxy-phenyl)-1,3-dioxolane, nonyl acid vanillylamide, 4-(1-menthoxy-methyl)-2-(3′-methoxy-4′-hydroxyphenyl)-1,3-dioxolane, ascorbic acid, black pepper, ginger, licorice, pilocarpine, affinin, spilanthol, bethanechol chloride, cayenne pepper, echinacea, verba santa, bay berry, sanguinarine, ginseng, kava, kudzu, capsaicin, zingerone, eugenol, piperine, and mixtures thereof.

11. Biofilm-responsive interproximal dental devices according to claim 4, wherein said sialagogue also imparts anti-inflammatory activity involving inhibiting cytokines, free radicals and prostaglandins.

12. A method for patient self-treatment for removing, disrupting and controlling biofilms and treating residual biofilms, while increasing and prolonging saliva flow comprising periodically introducing into interproximal spaces in the oral cavity, an interproximal dental device coated with biofilm-responsive, saliva soluble coatings comprising a substantially water-free emulsion having a surfactant continuous phase and a polydimethylsiloxane discontinuous phase, wherein said emulsion contains at least one sialagogue.

13. A method for patient self-treatment for dry mouth comprising periodically introducing into interproximal spaces in the oral cavity, an interproximal dental device coated with biofilm-responsive saliva soluble coatings comprising a substantially water-free emulsion having a surfactant continuous phase and a polydimethylsiloxane discontinuous phase, wherein said emulsion contains at least one sialagogue.

14. A method for manufacturing biofilm therapy interproximal dental devices coated with biofilm-responsive saliva soluble coatings comprising coating said devices with a saliva soluble coating containing an emulsion/sialagogue mixture with a coating process selected from the group of coating processes comprising: compression loading, injection loading, contact loading, dipping, spraying and combinations thereof.

15. A method according to claim 14, wherein SOFT ABRASIVES® particulate overcoating is included with coating the various interproximal devices.

16. Biofilm therapy dental devices according to claim 5, wherein said multifilament dental floss is a texturized multifilament dental floss.

17. Biofilm therapy dental devices according to claim 6, wherein said monofilament dental tape is a fibrillated, ultra-high molecular weight polyethylene dental tape.

18. Biofilm therapy dental devices according to claim 6, wherein said monofilament dental tape is a blown, high-density polyethylene dental tape.

19. Biofilm therapy dental devices according to claim 6, wherein said dental device is a proxy brush.

20. Biofilm therapy dental devices according to claim 6, wherein said dental device is a dental stimulator.

21. Biofilm therapy dental devices according to claim 6, wherein said dental device is a dental flosser.

22. Biofilm therapy dental devices according to claim 6, wherein said dental device is a dental massager.

23. Biofilm therapy dental devices according to claim 6, wherein said dental device is an interdental irrigator.

24. Biofilm therapy interproximal dental devices coated with a biofilm-responsive, saliva soluble coating comprising a substantially water-free emulsion having a surfactant continuous phase and a polydimethylsiloxane discontinuous phase, wherein said emulsion contains at least one sialagogue, wherein said saliva soluble coating is selected from the group consisting of high melt viscosity mixtures and emulsions; medium melt viscosity mixtures and emulsions; and low melt viscosity mixtures and emulsions, and combinations thereof.

25. Biofilm therapy interproximal dental devices coated with biofilm-responsive saliva soluble coatings according to claim 5, where said coatings contain aliphatic, long chain fatty alcohols suitable for controlling crystal formation in said coatings having the structural formula, ROH, wherein R represents a long chain alkyl group having between 20 and 30 carbon atoms.

26. Biofilm therapy interproximal dental devices according to claim 25, wherein said long chain fatty alcohol suitable for controlling crystal formation is selected from the group consisting of:

| | | |
|---|---|---|
| 1-decanol | 1-heptadecanol | 1-pentacosanol |
| 1 undecanol | 1-octadecanol | 1-hexacosanol |
| 1-dodecanol | 1-nonadecanol | 1-heptacosanol |
| 1-tetradecanol | 1-decosanol | 1-octacosanol |
| 1-pentadecanol | 1-henticosanol | 1-nonacosanol |
| 1-hexadecanol | 1-tricosanol | 1-triacosanol |
| 1-tetracosanol, | | |
| and mixtures thereof. | | |

27. Biofilm therapy interproximal dental devices according to claim 25 containing liquid surfactants represented by the general formula: 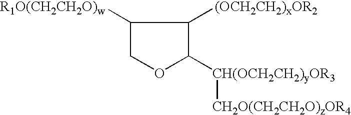
wherein R₁, R₂, R₃, R₄ and H or aliphatic acyl groups having from between about 10 and 30 carbon atoms, and the sum of w, x, y, and z is from between about 20 and about 80.

28. Biofilm therapy interproximal dental devices according to claim 5, wherein said surfactant continuous phase is selected from the group consisting of:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and polyoxyethylene compound having as a hydrophobe a polyoxypropylene polymer of at least 1200 molecular weight,
soap powder, and
mixtures thereof.

29. Biofilm therapy dental devices according to claim 2, overcoated with a soft abrasive overcoating.

30. Interproximal dental devices coated with a releasable sialagogue, wherein said devices are suitable for:
(a) controlling and/or disrupting and/or physically removing biofilms, while
(b) increasing and prolonging saliva flow.

31. The dental devices according to claim 30, wherein the releasable sialagogue further comprises a wax/sialagogue mixture.

32. The dental devices according to claim 30, wherein the releasable sialagogue further comprises a surfactant/sialagogue mixture

33. The dental devices according to claim 30, wherein the releasable sialagogue further comprises an emulsion/sialagogue mixture.
